(19) United States
(12) Patent Application Publication
(10) Pub. No.: US 2020/0347392 A1
(43) Pub. Date: Nov. 5, 2020

(54) NEXT GENERATION OF LIGHT-EMITTING PLANT LONGER DURATION AND BRIGHTER

(71) Applicant: MASSACHUSETTS INSTITUTE OF TECHNOLOGY, Cambridge, MA (US)

(72) Inventors: Michael Strano, Lexington, MA (US); Seonyeong Kwak, Cambridge, MA (US); Pavlo Gordiichuk, Cambridge, MA (US)

(73) Assignee: MASSACHUSETTS INSTITUTE OF TECHNOLOGY, Cambridge, MA (US)

(21) Appl. No.: 16/865,684

(22) Filed: May 4, 2020

Related U.S. Application Data
(60) Provisional application No. 62/843,550, filed on May 5, 2019.

(51) Int. Cl. C12N 15/82; A01H 1/06; A01G 7/00; F21S 11/00; F21K 2/06; F21V 33/00

(52) U.S. Cl. CPC C12N 15/825; A01H 1/06; A01G 7/00; B82Y 5/00; F21K 2/06; F21V 33/00; F21S 11/00

(57) ABSTRACT

A light emitting plant can include a long duration emissive material.

### CLAIM OF PRIORITY

This application claims priority to U.S. Provisional Patent Application No. 62/843,550, filed May 5, 2019, which is incorporated by reference in its entirety.

### FIELD OF INVENTION

This invention relates to light emitting plants.

### BACKGROUND

Plant genetic engineering is an important tool used in current efforts in crop improvement, pharmaceutical product biosynthesis and sustainable agriculture.

### SUMMARY OF THE INVENTION

In one aspect, a light emitting plant can include a plant structure and a light capacitor in a portion of the plant structure.

In another aspect, a plant can include a light generator, and a light capacitor for upconverting absorbed light to a wavelength absorbed by the light generator, wherein the light generator and the light capacitor are within a structure of the plant.

In another aspect, a method of generating light from a plant can include generating light in the plant with a light capacitor within the plant. In certain circumstances, the method can include storing energy from the light in the light capacitor; and releasing photons from the light capacitor, wherein the light capacitor is in a plant.

In certain circumstances, the light capacitor can scavenge additional energy from solar fluence, increasing and augmenting total light emission from the plant.

In certain circumstances, the light capacitor can be phosphorescent. For example, the light capacitor can be a phosphorescent nanoparticle or microparticle. In certain circumstances, the light capacitor can be a phosphorescent nanoparticle or microparticle including a strontium aluminate.

In certain circumstances, the light capacitor can be a coated particle. For example, the coated particle can be a silica coated particle. The silica coated particle can be a silica coated strontium aluminate. The silica coated strontium aluminate can be phosphorescent.

In certain circumstances, wherein the plant can include a second emissive component.

In certain circumstances, the light capacitor can be distributed inside plants leaves in spongy mesophyll region without penetration inside plants cell.

In certain circumstances, the light capacitor can be distributed inside the plant's stem.

In certain circumstances, the light capacitor can include a phosphorescent material.

The phosphorescent material can include a phosphor mineral. In certain circumstances, the phosphorescent material can have an emission lifetime of greater than 1 millisecond.

In certain circumstances, the phosphorescent material can emit green light.

In certain circumstances, the phosphorescent material can include a shell, for example, a silica shell.

In certain circumstances, the light capacitor can include an up-conversion material, such as, for example, a metal porphyrin and anthracene.

In certain circumstances, the method can include emitting light from a light generator.

In certain circumstances, the light generator can include luciferase-luciferin.

Other embodiments are described below and are within the claims.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic illustration of nanoparticles in a nanobionic light-emitting plant (left) and two light-emitting watercress plants illuminating a book (right).

FIGS. 2A-2B depict the design and Fabrication of Light Capacitor. FIG. 2A illustrates a comparison of light duration between high (4 μM) and low (0.2 μM) at a high concentration of PLGA-LH₂ (1 mM) and CS-CoA (625 μM). The model plot (red line), which accounted for the reaction rates and releasing kinetics of nanoparticles, which showed great fit with experimental data. The green dotted line illustrates the chemiluminescence decay can be achieved by light capacitors. FIG. 2B illustrates a luciferase-luciferin reaction, upconversion and light capacitor.

FIGS. 3A-3B depict light emission and its optimization in living plants. FIG. 3A illustrates a summary of maximum photons/sec (Iₘₐₓ) versus total duration of illumination (Tₘₐₓ) for different concentration of nanoparticles (n=3) in a plant tissue (V=2.5×10⁻² cm³). (α, β)=([SNP-Luc] μM, [PLGA-LH₂] mM). The error bars were calculated as a standard deviation of at least of triplicate. FIG. 3B illustrates a comparison of an estimated number of photons/sec from the light emitting plant (blue squares) to the maximum number of photons/sec calculated at current system (red and black lines).

FIG. 4A-4G depict a schematic image of the fabrication of colloidal stable phosphor nanoparticles. FIG. 4A depicts the strategy of reducing the size of Strontium Aluminate particles by wet milling, which results in a blue shift in the emission spectrum. SEM images of (FIG. 4B) the starting phosphor material, (FIG. 4C) wet milled materials, showing reduced sizes and blue shift in emission spectrum, and (FIG. 4D) Si/SiO₂ coated particles, showing the restoration of green emission light/a red shift. FIG. 4E depicts spectra of starting material, wet-milled (not modified with Si/SiO₂ shell) and wet-milled sample modified with Si/SiO₂ shell respectively. (F) Decay time as a function of phosphor consecrations, demonstrating that decay time undergo into saturation. (G) The relationship between measured saturation intensity of starting non-milled phosphor and the Si-coated milled phosphor samples of different concentrations. I is phosphorescent intensity measured at 530 nm wavelength under continuous excitation with 400 nm light-emitting diode (LED) with 100 μW power.

FIG. 5A-5F depict characterization of ultrasonic milled samples. FIG. 5A illustrates an SEM images of wet milled strontium aluminate (481.5±26.0) particles before ultra-sonication (top panel) and after one hour of sonication 51.96.4 nm (bottom panel). FIG. 5B illustrates histograms of particle size measured from SEM images for samples treated with ultra-sonication for 0, 1, 5, 10 and 20 min. FIG. 5C illustrates size distribution of milled strontium aluminate samples centrifuged for 30, 40, 50, 60, 70, 80, 90 and 100 min obtained by single particle tracking. FIG. 5D illustrates reduction of milled particles sizes plotted against the ultra-sonication time and the reduction of absorption at the fixed 400 nm wavelength. FIG. 5E illustrates absorption spectra of the samples against the centrifugation time. FIG. 5F illustrates size dependent PL for samples with different centrifugation time.

FIG. 6A-6F depict size and PL characterization of Si-coated strontium aluminate particles. FIG. 6A illustrates particle size distribution of each sample collected at different time of integrated centrifuging, which was obtained by the single particle tracking. FIG. 6B illustrates the particle diameter plotted as a function of centrifugation time. FIG. 6C illustrates absorption spectra of sorted samples. FIG. 6D illustrates photoluminescence (PL) changes of nanoparticles recorded after 1, 2, 4, 6 and 8 minutes of centrifuging which indicate a clear shift toward the IR region. FIG. 6E illustrates a TEM image of the nanoparticles after 2 min of centrifuging. FIG. 6F illustrates an SEM images of the sorted Si-coated nanoparticles.

FIG. 7A-7C depict photophysical properties of both milled and Si-coated milled phosphor. FIG. 7A illustrates photoluminescence images of strontium aluminate nanoparticles, showing dependence of emission color on particle sizes. FIG. 7B illustrates calculated decay constants as a function of particle size. FIG. 7C illustrates size-dependent afterglow lifetime of Si-coated strontium aluminate particles.

FIG. 8A depicts schematic image of milling (i), Si/SiO₂ coating (ii) and application for infiltration in to plants leaves of SA particles. FIG. 8B depicts SEM images of commercially available SA before milling and after milling with Si/SiO₂ coating step. FIG. 8C depicts propagation of infiltration solution in the horizontal direction from the contact points realized with tipless syringe. Proper infiltration can result in almost complete infiltration of watercress leaf. Scale bar: 1 cm. FIG. 8D depicts integrated centrifuging strategy of Si/SiO₂ mSA for collecting individual pellets of particular size.

FIGS. 9A-9F illustrate chlorophyll concentration measurements (in SPAD units) of infiltrated watercress leaves with (FIG. 9A) raw SA material, (FIG. 9B) milled SA (mSA) material at pH 14, (FIG. 9C) mSA at pH 7, (FIG. 9D) raw material coated with Si/SiO₂, (FIG. 9E) mSA at pH 7, (FIG. 9F) Si/SiO₂ coated of mSA at pH 7. All samples were at 50 mg/ml (red), 25 mg/ml (purple), 10 mg/ml (green), 5 mg/ml (brown), 1 mg/ml (blue) concentrations respectively.

FIGS. 10A-10C depict assimilation curves showing net CO₂ assimilation rate as function of internal CO₂ concentrations (Ci) in watercress modified with Si/SiO₂ mSA particles (FIG. 10A), in watercress leaf modified with just HEPES buffer (FIG. 10B, Control 1), and non-modified watercress leaf (FIG. 10C, Control 2). Dotted lines show a linear connection between points.

FIGS. 11A-11E depict SEM images of sorted samples by centrifuging resulting in different sizes of (FIG. 11A) 1087.4±414.9 nm for 500 rpm speed, (FIG. 11B) 899.0±358.9 nm at 1000 rpm speed, (FIG. 11C) 651.9±292.1 nm at 2000 rpm speed, (FIG. 11D) 441.9279.6 nm at 3000 rpm speed and (FIG. 11E) 386.8180.6 nm respectively. Measurements were performed in Image.

FIG. 12 depicts confocal images of infiltrated watercress leaves with S3 Si/SiO₂ mSA (panels A-C) samples and S4 Si/SiO₂ mSA (D-F) samples respectively. Panel A shows phosphorescence of S3 mSA particles inside watercress leaves. Panel B shows autofluorescence of chlorophylls. Panel C shows an overlay of Panel A and Panel B. Panel D shows phosphorescence of S4 mSA particles inside watercress leaves. Panel E shows autofluorescence of chlorophylls. Panel F shows an overlay of Panel D and Panel E.

FIGS. 13A-13D depict cryo scanning electron microscopy measurements on watercress leaves in cross section. FIG. 13A shows infiltrated watercress leaf infiltrated with S3 sample. FIG. 13B shows a detailed zoom of image of Si/SiO₂ mSA particles agglomerated on cells walls. FIG. 13C shows infiltrated watercress leaves with HEPES buffer. (E) Non-modified watercress leaves. FIG. 13D shows the non-infiltrated plant.

FIG. 14A-14D depict the following. FIG. 14A illustrates infiltrated watercress leaves with different sizes of Si/SiO₂ coated mSA particles named S, S2, S3, S4 and S5. FIG. 14B illustrates corresponding absorption spectrums of S-S5 samples. FIG. 14C illustrates intensity decay curves recorded with camera each 1 min under exposure of 30 sec for samples S1-S5 respectively under 30 s charging with 400 nm LED of 10 W power. FIG. 14D illustrates intensity decay of samples S3 in watercress leaf under triple charging and decay intensity measurements.

FIGS. 15A-15E depict the following. FIG. 15A shows infiltrated watercress with sample S3. FIG. 15B shows infiltrated Basil with samples S3. FIG. 15C shows infiltrated Gerbera Daisy with sample S3. FIG. 15D shows phosphorescence intensity decay in all three plants measured over 1 hour under 30 s exposure each 1 min demonstrating decay time of 1.12±0.07, 1.82±0.07 and 7.360.33 min respectively. FIG. 15E shows stability of the same watercress leaf during one week marked as day 0 and day 7 respectively.

FIG. 16 depicts TEM images of strontium aluminate starting material shipped from Luminova company with the average size of 3 μm.

FIG. 17 depicts PL spectra of strontium aluminate powder deposited from solution on glass substrate and dried. Samples were excited from the side with hand Mercury UV lamp.

FIG. 18 depicts decay time of strontium aluminate sample dispersed in water under continuous stirring over a time after different time excitation for 10, 30 and 60 min.

FIG. 19 depicts a spectrum of LED charging source shipped from Thorlabs part number: M365L2.

FIGS. 20A-20B depict emission light measurements from phosphor Starting material (FIG. 20A) of different concentration (under 365 nm LED excitation) and Si-coated Milled samples (FIG. 20B).

FIG. 21 depicts TEM images of Si nanoparticles created as a secondary product during Strontium Aluminate particles coating.

FIG. 22 depicts TEM EDX elemental analysis maps shows composition of nanoparticles containing Si and O chemical elements respectively, where no signatures of Strontium and Aluminum were observed.

FIG. 23 depicts a plot describes relationship between concentration and measure corresponding optical density of samples (O.D.).

FIG. 24 depicts a plot describes relationship between concentration and measure corresponding optical density of samples (O.D.).

FIG. 25 depicts photoluminescence spectra of 1 mg/ml samples collected at different centrifuging time of 1, 2, 4, 6 and 8 min, respectively.

FIG. 26 depicts green light emission from watercress leaves recorded with a camera after short (5 s) exposure to blue light-emitting diode. Light intensity captured directly after excitation (0 min) and 1 min demonstrates monotonic decay over a time.

FIG. 27 depicts modified single leaf and corresponding stem of watercress plant showed characteristic emission at the beginning (0 min) and 1 min, a clear indication of a single leaf and single stem modification.

### DETAILED DESCRIPTION

Nanoparticle-mediated transformation represents a promising approach for plant genetic engineering. Although nanoparticles have been widely studied to deliver biomolecules to animal cells and tissues in recent years, their use in plants is limited due to their potential toxicity and limited knowledge of how they interact with plant biological membranes and the multilayered cell wall. The biolistic approach has previously been employed to deliver mesoporous silica nanoparticles containing genetic materials and chemicals past the rigid cell wall into the cytosol of plant protoplasts and seedlings. However, nanoparticle-mediated gene delivery into a specific organelle of mature plants without external mechanical aid has not been demonstrated. Specifically designed nanoparticles, including single-walled carbon nanotubes (SWNTs), can traverse the rigid plant cell walls, membranes and even the double lipid bilayers of chloroplasts before they become kinetically trapped within the chloroplasts. This passive nanoparticle uptake mechanism was described using a mathematical model called Lipid Envelope Exchange Penetration (LEEP), whereby the ability of nanoparticles to penetrate the cell membrane and the double lipid bilayer of chloroplasts is governed primarily by nanoparticle size and surface charge. Based on this mechanism, the tunable physical and optical properties of nanomaterials can be leveraged to optimize the passive delivery of biological cargoes across many plant barriers that have hitherto been difficult to access. The study is theoretically not limited to SWNTs. SWNTs were selected out of various nanomaterials because SWNTs have attracted considerable interest as nanocarriers for drug and gene delivery due to their high aspect ratio and large surface area for chemical modification. However, existing applications of SWNTs in plants were primarily limited to studies of SWNTs transport in plant tissues or cells, and none of the work explored the possibility of utilizing SWNTs as nanocarriers for gene delivery into specific plant organelles. Chitosan-wrapped single-walled carbon nanotubes (CS-SWNTs) have been shown to possess sufficiently high surface charge to allow them to passively penetrate the plant membrane and double lipid bilayers of chloroplasts. For successful gene delivery, the pDNA has to be condensed by chitosan-functionalized SWNTs, safely transported to the chloroplasts after crossing various plant membranes, intracellularly detached and transiently expressed within the chloroplast stroma. The potential use of chitosan as a polycationic gene carrier for plant transformation is implied by its capability to form a complex with negatively charged pDNA via electrostatic interactions, protecting pDNA from nuclease degradation. In addition, chitosan is a biodegradable polysaccharide, abundant in nature and non-toxic to plant systems.

A light capacitor, as described herein, is a composition that absorbs light and re-emits light. The light capacitor can be a fluorescent composition or a phosphorescent composition. The light capacitor can convert a wavelength of light that is absorbed and emit light of a wavelength that can be used to perform other tasks, for example, be absorbed by another component in a plant. The light capacitor can allow the captured light to be used in a plant at a later time that at the time of initial irradiation. For example, the light capacitor can upconvert a wavelength of light. For example, the up conversion can be from a green to a near visible wavelength. A bright nanobionic light-emitting plants (LEPs) can use a light-capacitor, for example, when the plant is infiltrated with phosphor particles inside plant leaves that capture light and which can be used as an afterglow in the dark.

The phosphor can be a nanophosphor composition. For example, the phosphor can be a strontium aluminate, a doped ytrrium oxide composition, a porphyrin-containing composition or a luciferase-luciferin reaction. The phosphor can be an up-conversion material, for example, a rare earth halide nanoparticles, lanthanide-doped nanoparticles, or semiconductor nanoparticles. The up-conversion material can convert 980 nm infrared light to 600 nm visible light; green light to blue light; or blue light to ultraviolet.

As described herein, a light emitting plant can include a plant structure and a light capacitor in a portion of the plant structure. The light capacitor can be phosphorescent, for example, a phosphorescent microparticle or nanoparticle, such as a strontium aluminate. The light capacitor can be a coated particle, for example, a silica-coated particle. The light capacitor can scavenge additional energy from solar fluence, increasing and augmenting total light emission from the plant, for example, by energy transfer to a second emissive component. For example, a light capacitor can upconvert absorbed light to a wavelength absorbed by the light generator when the light generator and the light capacitor are within a structure of the plant. In certain embodiments, the light capacitor can be distributed inside plants leaves in spongy mesophyll region without penetration inside plants cell, inside the plant's stem. The up-conversion material can include a metal porphyrin and anthracene and the light generator can include luciferase-luciferin.

As described herein, a wild type plant can be to grow and thrive outdoors, a functional plant or tree in the wild, already adapted to its local natural environment. This is not a reference to new organisms such as GMO plants or to engineer genetically pliable Tobacco or Arabidopsis plants in the laboratory. This ultimately allows us to use infiltrated nanoparticles to engineer new features and functions in a living plant. This idea was first introduced in a Nature Materials by the Strano lab in 2014 with some progress on nanoparticle stabilization made the year earlier and recently, a living, wild-type plant was shown to be capable of detecting groundwater contamination and infrared communication in a Nature Materials. See, for example, Giraldo, J. P. et al. Plant nanobionics approach to augment photosynthesis and biochemical sensing. Nature Materials 13, 400-408 (2014), Boghossian, A. A. et al. Application of Nanoparticle Antioxidants to Enable Hyperstable Chloroplasts for Solar Energy Harvesting. Advanced Energy Materials 3, 881-893 (2013), and Wong, M. H. et al. Nitroaromatic detection and infrared communication from wild-type plants using plant nanobionics. Nature Materials 16, 264-272 (2017), each of which is incorporated by reference in its entirety. For the first time, the ability to predict and control the localization and trafficking of designer nanoparticles to specific plant tissues, cells and organelles has been developed. To elucidate this, Lipid Envelope Exchange Penetration (LEEP) mechanism was developed, which describes interactions between charged nanoparticles and the surface charges on the chloroplast membrane and irreversible trap of the lipid-wrapped nanoparticles within the chloroplast. See, for example, Wong, M. H. et al. Lipid Exchange Envelope Penetration (LEEP) of Nanoparticles for Plant Engineering: A Universal Localization Mechanism. Nano Lett. 16, 1161-1172 (2016), which is incorporated by reference in its entirety. Furthermore, Pressurized Bath Infusion of Nanoparticles (PBIN) is developed to deliver mixture of nanoparticles to the entire living plant, well described using a nanofluidic mathematical model. See, for example, Kwak, S.-Y. et al. A Nanobionic Light-Emitting Plant. Nano Lett. 17, 7951-7961 (2017), which is incorporated by reference in its entirety. To realize the vision of a light emitting plant, these techniques provide new opportunities to control the location and concentrations of light generating reactions within the living wild-type plant.

The concept of a light-emitting plant, or plant exhibiting chemiluminescence powered from its own stored chemical energy, offers promise to advance off grid illumination and other autonomous photonic applications. Recent efforts using nanotechnology and specifically plant nanobionics have introduced high performing light emitting plants incorporating chemically interacting nanoparticles delivered into specific locations within plant tissues. In this work, introduce and investigate an additional nanoparticle designed to augment plant light emission in the form of silica coated strontium aluminate nanoparticles as nanophosphore elements. These nanoparticles can adsorb and re-emit generated light at longer times, increasing the duration of light emission. Moreover, such nanophosphores can also scavenge additional energy from solar fluence, increasing and augmenting total light emission from the plant. Infiltrated strontium aluminate particles showed homogeneous distribution inside plants leaves in spongy mesophyll region without penetration inside plants cell, preserving their intact structure, as well as efficient particles infiltration deep into the plant's stem. Performed photosynthetic activity on modified plants confirmed their intact functionality with minor reduction of chlorophyll amount comparable to non-modified plants related to mechanical damaging during particles infiltration. Studied post excitation emission showed homogeneous afterglow from plants leaves modified with 651.9±292.1 nm sized particles, where the infiltrated leaves of watercress, daisy and basil possessed intensity decay time of 1.12±0.07, 1.82±0.07 and 7.36±0.33 min respectively indicating their selective permeability of to a certain particle size and stability for more than one week.

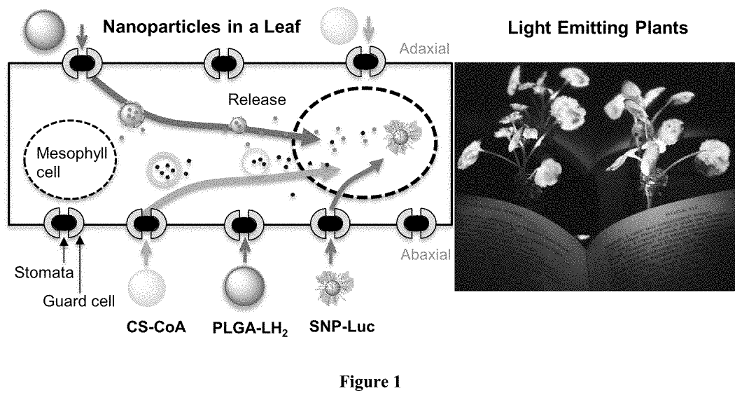

As shown in FIG. 1, a schematic illustration of nanoparticles in a nanobionic light-emitting plant (left) and two light-emitting watercress plants can illuminate a book (right).

In general, the invention relates to nanoparticle-modified plants.

1. Design and Fabrication of Light Capacitor

In chemiluminescence decay kinetics, a sharp drop in the light intensity in few minutes and the decreased intensity continued over several hours has been observed. Although the incubation time and the use of Chitosan nanoparticles with Coenzyme A (CS-CoA) can be extended the duration to nearly 4 hours (up to 8.5 hours), a further extension of the light duration by storing the initial burst of energy and releasing the photons slowly over time with the use of a light capacitor. In order to design the light capacitor, a phosphor mineral that includes phosphorescent materials can be chosen which show a slow decay in brightness (>1 ms). A mineral that emits green light can be chosen because green light is barely absorbed by chlorophyll pigments in plant tissues. Materials of this type can exhibit persistent luminescence that is observable by eye for several hours after excitation and is highly resistant to photobleaching. See, for example, Matsuzawa, T., Aoki, Y., Takeuchi, N. & Murayama, Y. A New Long Phosphorescent Phosphor with High Brightness, SrAl2 O 4 Eu2+, Dy3+. J. Electrochem. Soc. 143, 2670-2673 (1996), and Swart, H. C., Terblans, J. J., Ntwaeaborwa, O. M., Kroon, R. E. & Mothudi, B. M. PL and CL degradation and characteristics of SrAl204: Eu2+,Dy3+ phosphors. Physica B: Condensed Matter 407, 1664-1667 (2012), each of which is incorporated by reference in its entirety. To improve biocompatibility and water stability of the phosphors, the Stöber method can be used to make the particles with a slight modification to form a silica shell around the phosphors. Since the phosphors are excited by ultraviolet (UV) and near visible light (λₑₓ=200-400 nm) rather than excited directly by luciferase-luciferin reaction (λₑₘ=560 nm), noncoherent sensitized green-to-near-visible or -UV up-conversion materials to apply to our system can be studied. One promising candidate is metal coordinated porphyrin as the triplet sensitizer (λₑₓ=547 nm, green) and anthracene as the energy acceptor/annihilator (λₑₘ=380 nm, near visible). See, for example, Deng, F., Blumhoff, J. & Castellano, F. N. Annihilation Limit of a Visible-to-UV Photon Upconversion Composition Ascertained from Transient Absorption Kinetics. J. Phys. Chem. A 117, 4412-4419 (2013), which is incorporated by reference in its entirety. A regenerative photochemical process, sensitized triplet-triplet annihilation (TTA), can be achieved in the frequency upconversion of light. See, for example, Singh-Rachford, T. N. & Castellano, F. N. Photon upconversion based on sensitized triplet-triplet annihilation. Coordination Chemistry Reviews 254, 2560-2573 (2010), and Monguzzi, A., Tubino, R., Hoseinkhani, S., Campione, M. & Meinardi, F. Low power, non-coherent sensitized photon up-conversion: modelling and perspectives. Phys. Chem. Chem. Phys. 14, 4322-4332 (2012), each of which is incorporated by reference in its entirety. An alternative approach is to utilize upconverting ceramic nanoparticles that emits UV upconversion luminescence induced by 532 nm wavelength. See, for example, Qin, F. et al. Ultraviolet and violet upconversion luminescence in Ho3+-doped Y₂O₃ ceramic induced by 532-nm CW laser. Journal of Alloys and Compounds 509, 1115-1118 (2011), which is incorporated by reference in its entirety. The light capacitor particles can consist of multi-layers that absorb the green light generated by luciferase-luciferin reaction, upconvert this visible light to UV or near visible, and re-emit visible light (phosphorescence). The integration of light capacitor in the plant nanobionic system can result in a significant increase in the total integrated number of photons released from the plant.

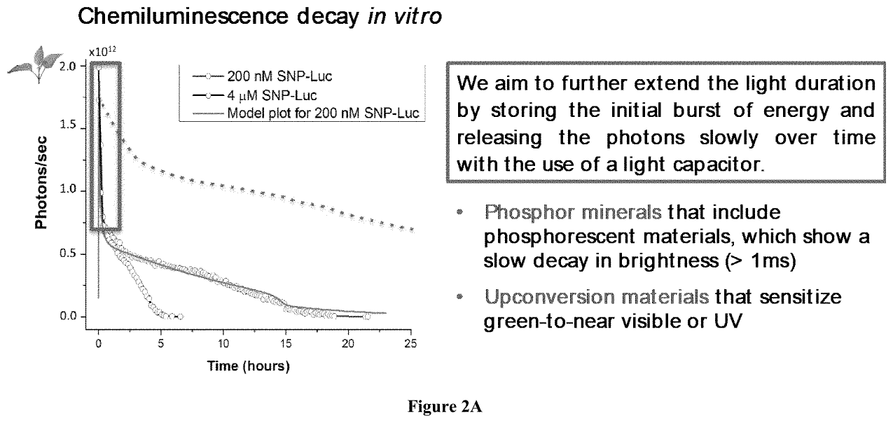

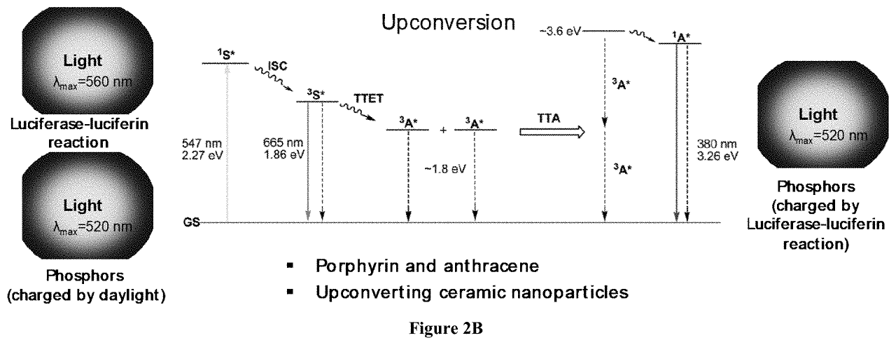

A shown in FIGS. 2A-2B, the design and fabrication of a light capacitor can include: (FIG. 2a) Comparison of light duration between high (4 μM) and low (0.2 μM) at a high concentration of PLGA-LH₂ (1 mM) and CS-CoA (625 μM). The model plot (red line), which accounted for the reaction rates and releasing kinetics of nanoparticles, which showed great fit with experimental data. The green dotted line illustrates the chemiluminescence decay can be achieved by light capacitors. In FIG. 2B, an illustration of luciferase-luciferin reaction, upconversion and light capacitor is shown.

2. Scale-Up of the Nanobionic Light-Emitting Plant

A method of infusion using stomatal pores within the leaves termed Pressurized Bath Infusion of Nanoparticles (PBIN) has been developed. PBIN is able to simultaneously infiltrate the nanoparticle mixture into a whole plant but a scalable enclosure is needed to apply PBIN to large trees or many plants. The wetting properties of the particle solution can be lowered such that spontaneous, atmospheric delivery into the plant is possible. A nanofluidic model describes how PBIN works by supplying an external pressure against the internal microchannels within the leaf spongy mesophyll, generating an inward flow through the stomatal pores. The net inward velocity is dictated by the sum of the capillary forces, viscous drag, resistance from trapped air compression, and the applied PBIN force. In a scale-up infiltration method, the applied PBIN force will be same as atmospheric pressure. Since the contact angle of water drop on the leaf surface (θ) is critical factor, surfactants can be used to temporally modify the leaf surface. Various non-ionic surfactants can be explored, such as sugar moiety containing surfactants to minimize effects on plant heath. A standardized way to apply nanoparticle mixtures to plants by contact-based (e.g. paint), or water-based (e.g. spray) or air-based (e.g. pressurize) can be developed.

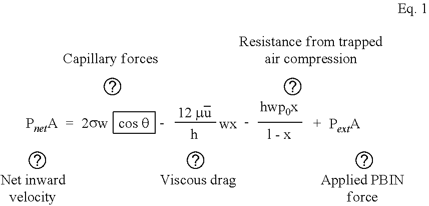

Water-based solutions of co-enzymes can be introduced to living trees. The development of scale-up strategies will be studied through a scenario-based process, driven by the specific light brightness and duration targets achieved through the light capacitor experiments as well as their projections in the mathematical model of the light-emitting plant. Scale-up for safe and sustainable light emitting particle delivery methods can be achieved through common practices such as drip irrigation or hose-based watering regimes at the tree scale. As with the light emitting plant, an incremental approach will be studied, which can accommodate scale up for tree light duration periods that range from multiple hours to days.

Nanobionic Light Emitting Plants with Transient Genetic Engineering of the Chloroplast

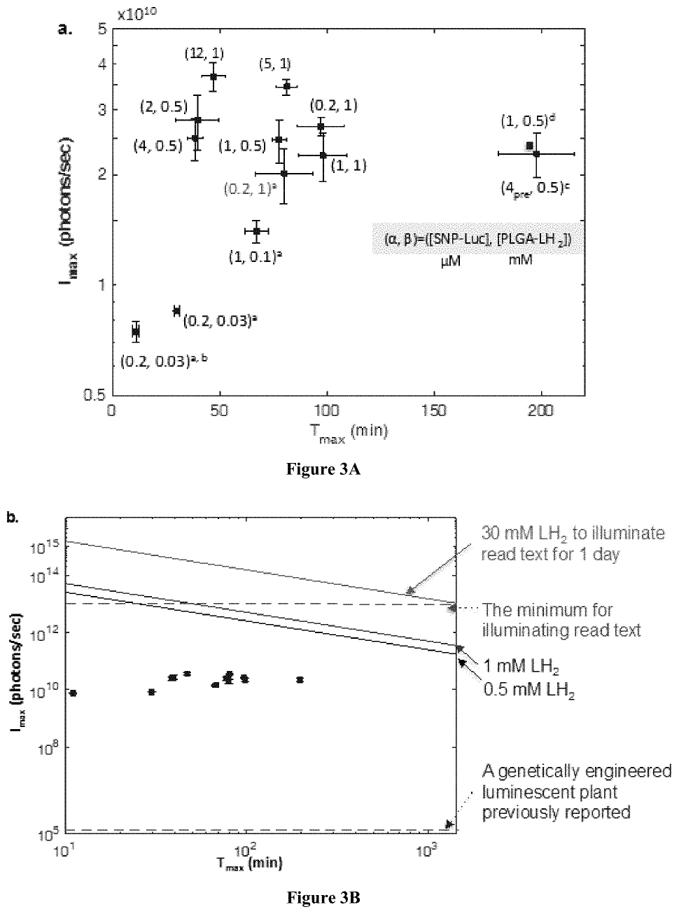

The precisely designed nanoparticles with a sustained rate of chemical release, the controlled sizes and formulate can extend the chemiluminecent lifetime and intensity in living plant systems. It appears to be critical to keep the chemiluminescent reactive zones continuously supplied with reagents (FIG. 3A). The maximum possible photons available for emission in the plant are plotted after accounting for tissue reabsorption, concentration of the limiting reagent (FIG. 3B). Iₘₐₓ (the highest light intensity) given Tₘₐₓ (the light duration) show that the plants described here are >10⁵ brighter than a genetically engineered Nicotiana tabacum plant with >10 times longer. See, for example, Krichevsky, A., Meyers, B., Vainstein, A., Maliga, P. & Citovsky, V. Autoluminescent Plants. PLoS ONE 5, e15461 (2010), which is incorporated by reference in its entirety. The model plot suggests that the Iₘₐₓ values are 2-3 orders of magnitude below the predicted maximum (FIG. 3B). Higher intensities are possible by enhancing the permeability of SNP-Luc into the mesophyll cells where ATP exists in high concentration. At longer times, increasing the loading of LH₂ and CoA within their respective nanoparticles to account for the extended flux can eliminate these limitations. If both are achieved, a light duration of more than 17 days (417 hours) at 2·10¹⁰ photons/sec can be achieved. This can be even longer with the light capacitors. Further optimization of the infusion and particle concentration will yield further improvement to both the intensity and duration of light-emitting plant. The optimally designed nanoparticles will be infused into living plants to compare the actual and the predicted lifetime and intensity of chemiluminescence. Together with our mathematical model to validate in vitro reaction, an integrated mathematical model of chemically interacting nanoparticles within the biochemical environment of the plant can be developed. In vivo microscopy and hyperspectral imaging can be used to determine transport rates in real time as direct comparison to the mathematical model developed.

In FIGS. 3A-3B, light emission and its optimization in living plants is shown. FIG. 3A depicts a summary of maximum photons/sec (Iₘₐₓ) versus total duration of illumination (Tₘₐₓ) for different concentration of nanoparticles (n=3) in a plant tissue (V=2.5×10⁻² cm³). (α, β)=([SNP-Luc] μM, [PLGA-LH₂] mM). The error bars were calculated as a s.d. of at least of triplicate. FIG. 3B depicts a comparison of estimated number of photons/sec from the light emitting plant (blue squares) to the maximum number of photons/sec calculated at current system (red and black lines).

Despite of all the efforts, both limited lifetime of luciferase activity and limited amount of luciferin in plant systems are encountered. Luciferin-regenerating enzyme (LRE) can contribute to recycling of D-luciferin, which increase luciferase-luciferin light output. See, for example, Hemmati, R. et al. Luciferin-Regenerating Enzyme Mediates Firefly Luciferase Activation Through Direct Effects of D-Cysteine on Luciferase Structure and Activity. Photochemistry and Photobiology 91, 828-836 (2015), which is incorporated by reference in its entirety. Therefore, chloroplast-selective nanoparticle-mediated transient genetic engineering technique (organelle-selective gene delivery and expression can be used for expression in the chloroplast in planta using chitosan-complexed single-walled carbon nanotube carriers), to produce luciferase and LRE in plants. Chloroplast transformation offers advantages over conventional nuclear transformation technologies and thus represents a viable alternative approach for plant genetic engineering. See, for example, Fuentes, P., Armarego-Marriott, T. & Bock, R. Plastid transformation and its application in metabolic engineering. Curr. Opin. Biotechnol. 49, 10-15 (2018), and Jin, S. & Daniell, H. The Engineered Chloroplast Genome Just Got Smarter. Trends in Plant Science 20, 622-640 (2015), each of which is incorporated by reference in its entirety. Due to maternal inheritance of plastid genomes in most higher plants, chloroplast transformation provides a level of containment that rarely leads to genetic outcrossing of transgenes. See, for example, Khan, M. S., Kanwal, B. & Nazir, S. Metabolic engineering of the chloroplast genome reveals that the yeast ArDH gene confers enhanced tolerance to salinity and drought in plants. Front. Plant Sci. 6, 311 (2015), and Scott, S. E. & Wilkinson, M. J. Low probability of chloroplast movement from oilseed rape (Brassica napus) into wild Brassica rapa. Nature Biotechnology 17, 390-392 (1999), each of which is incorporated by reference in its entirety. Since the plastid genome is highly polyploid, transformation of chloroplasts can lead to extraordinarily high levels of foreign protein production by introducing thousands of copies of foreign genes per plant cell. See, for example, Cosa, B. D., Moar, W., Lee, S.-B., Miller, M. & Daniell, H. Overexpression of the Bt cry2Aa2 operon in chloroplasts leads to formation of insecticidal crystals. Nature Biotechnology 19, 71-74 (2001), which is incorporated by reference in its entirety. In addition, the reduced risk of mammalian viral contaminants and the ability of chloroplasts to fold human proteins has enabled high-yield production of human therapeutics. See, for example, Staub, J. M. et al. High-yield production of a human therapeutic protein in tobacco chloroplasts. Nature Biotechnology 18, 333-338 (2000) and Millin, A. F.-S., Castel, A. M., Miller, M. & Daniell, H. A chloroplast transgenic approach to hyper-express and purify Human Serum Albumin, a protein highly susceptible to proteolytic degradation. Plant Biotechnology Journal 1, 71-79 (2003), each of which is incorporated by reference in its entirety. Chloroplasts withstand stressful conditions such as high salt or drought, thereby the integrity of the products derived from chloroplasts transformation can be better preserved. See, for example, Khan, M. S., Kanwal, B. & Nazir, S. Metabolic engineering of the chloroplast genome reveals that the yeast ArDH gene confers enhanced tolerance to salinity and drought in plants. Front. Plant Sci. 6, 311 (2015), which is incorporated by reference in its entirety. Transient expression of foreign proteins can produce high yields of the desired proteins in a relatively short period of time (days) whereas stable expression requires longer development time (months) and is limited to a few species. See, for example, Canto, T. in Advanced Technologies for Protein Complex Production and Characterization 896, 287-301 (Springer, Cham, 2016), which is incorporated by reference in its entirety. Together the nanoparticle optimization and light capacitors, the transient expression of firefly luciferase and LRE in plants can be substantially extends the chemiluminescence lifetime.

As used herein, the term “nanoparticle” refers to articles having at least one cross-sectional dimension of less than about 1 micron. A nanoparticle can also be referred to as a “nanostructure.” A nanoparticle can have at least one cross-sectional dimension of less than about 500 nm, less than about 250 nm, less than about 100 nm, less than about 75 nm, less than about 50 nm, less than about 25 nm, less than about 10 nm, or, in some cases, less than about 1 nm. Examples of nanoparticle include nanotubes (e.g., carbon nanotubes), nanowires (e.g., carbon nanowires), graphene, and quantum dots, among others. In some embodiments, the nanoparticle can include a fused network of atomic rings, the atomic rings comprising a plurality of double bonds.

A nanoparticle can be a photoluminescent nanoparticle. A “photoluminescent nanoparticle,” as used herein, refers to a class of nanoparticles that are capable of exhibiting photoluminescence. In some cases, photoluminescent nanoparticles can exhibit fluorescence. In some instances, photoluminescent nanoparticles exhibit phosphorescence. Examples of photoluminescent nanoparticles suitable for use include, but are not limited to, single-walled carbon nanotubes (SWCNTs), double-walled carbon nanotubes (DWCNTs), multi-walled carbon nanotubes (MWCNTs), semi-conductor quantum dots, semi-conductor nanowires, and graphene, among others. The photoluminscent nanoparticle can include a phosphor material.

A variety of nanoparticles can be used. Sometimes a nanoparticle can be a carbon-based nanoparticle. As used herein, a “carbon-based nanoparticle” can include a fused network of aromatic rings wherein the nanoparticle includes primarily carbon atoms. In some instances, a nanoparticle can have a cylindrical, pseudo-cylindrical, or horn shape. A carbon-based nanoparticle can include a fused network of at least about 10, at least about 50, at least about 100, at least about 1000, at least about 10,000, or, in some cases, at least about 100,000 aromatic rings. A carbon-based nanoparticle may be substantially planar or substantially non-planar, or may include a planar or non-planar portion. A carbon-based nanoparticle may optionally include a border at which the fused network terminates. For example, a sheet of graphene includes a planar carbon-containing molecule including a border at which the fused network terminates, while a carbon nanotube includes a non-planar carbon-based nanoparticle with borders at either end. In some cases, the border may be substituted with hydrogen atoms. In some cases, the border may be substituted with groups comprising oxygen atoms (e.g., hydroxyl).

In some embodiments, a nanoparticle can include or be a nanotube. The term “nanotube” is given its ordinary meaning in the art and can refer to a substantially cylindrical molecule or nanoparticle including a fused network of primarily six-membered rings (e.g., six-membered aromatic rings). In some cases, a nanotube can resemble a sheet of graphite formed into a seamless cylindrical structure. It should be understood that a nanotube may also include rings or lattice structures other than six-membered rings. Typically, at least one end of the nanotube may be capped, i.e., with a curved or non-planar aromatic group. A nanotube may have a diameter of the order of nanometers and a length on the order of microns, tens of microns, hundreds of microns, or millimeters, resulting in an aspect ratio greater than about 100, about 1000, about 10,000, or greater. In some embodiments, a nanotube can have a diameter of less than about 1 micron, less than about 500 nm, less than about 250 nm, less than about 100 nm, less than about 75 nm, less than about 50 nm, less than about 25 nm, less than about 10 nm, or, in some cases, less than about 1 nm.

In some embodiments, a nanotube may include a carbon nanotube. The term “carbon nanotube” can refer to a nanotube including primarily carbon atoms. Examples of carbon nanotubes can include single-walled carbon nanotubes (SWNTs), double-walled carbon nanotubes (DWNTs), multi-walled carbon nanotubes (MWNTs) (e.g., concentric carbon nanotubes), inorganic derivatives thereof, and the like. In some embodiments, a carbon nanotube can be a single-walled carbon nanotube. In some cases, a carbon nanotube can be a multi-walled carbon nanotube (e.g., a double-walled carbon nanotube).

In some embodiments, a nanoparticle can include non-carbon nanoparticles, specifically, non-carbon nanotubes. Non-carbon nanotubes may be of any of the shapes and dimensions outlined above with respect to carbon nanotubes. A non-carbon nanotube material may be selected from polymer, ceramic, metal and other suitable materials. For example, a non-carbon nanotube may include a metal such as Co, Fe, Ni, Mo, Cu, Au, Ag, Pt, Pd, Al, Zn, or alloys of these metals, among others. In some instances, a non-carbon nanotube may be formed of a semi-conductor such as, for example, Si. In some cases, a non-carbon nanotube may include a Group II-VI nanotube, wherein Group II includes Zn, Cd, and Hg, and Group VI includes O, S, Se, Te, and Po. In some embodiments, a non-carbon nanotube may include a Group III-V nanotube, wherein Group III includes B, Al, Ga, In, and Tl, and Group V includes N, P, As, Sb, and Bi. As a specific example, a non-carbon nanotube may include a boron-nitride nanotube. In other embodiments, the nanoparticle can be a ceramic, for example, a metal oxide, metal nitride, metal boride, metal phosphide, or metal carbide. In this example, the metal can be any metal, including Group I metal, Group II metal, Group III metal, Group IV metal, transition metal, lanthanide metal or actinide metal. For example, the ceramic can include one or more of metal, for example, Li, Na, K, Rb, Cs, Be, Mg, Ca, Sr, Ba, Sc, Y, La, Ce, Pr, Nd, Pm, Sm, Eu, Gd, Tb, Dy, Ho, Er, Tm, Yb, Lu, Ti, Zr, Hf, V, Nb, Ta, Cr, Mo, W, Mn, Tc, Re, Fe, Ru, Os, Co, Rh, Ir, Ni, Pd, Pt, Cu, Ag, Su, Zn, Cd, Hg, Al, Ga, In, Tl, Si, Ge, Sn, Pb or Bi.

The nanoparticle can be a phosphor nanoparticle. The phosphor nanoparticle can include a phosphorescent material. The phosphorescent material can be a photoluminescent material that has a slow decay rate, for example, a decay rate of greater than 1 ms. A phosphor material can be an organic or an inorganic material. The inorganic material can include an emissive trap or metal atom. The emissive metal atom can be a transition metal element or rare earth element.

In some embodiments, the nanoparticle can be coated. The coating can be an inorganic coating or an organic coating, or a combination thereof. The inorganic coating can include a metal oxide. The inorganic coating can include a silicon oxide, a titanium oxide, a zirconium oxide, or a combination thereof. For example, the coating can include a silicon/SiO₂.

In some embodiments, the nanoparticle can be a conjugate. For example, the nanoparticle can be associated with a second nanoparticle or molecule, or a combination thereof. The molecule can be a protein, for example, a fluorescent protein. The nanoparticle can be associated with the second nanoparticle or molecule by an ionic or covalent linkage.

In some embodiments, a nanotube may include both carbon and another material. For example, in some cases, a multi-walled nanotube may include at least one carbon-based wall (e.g., a conventional graphene sheet joined along a vector) and at least one non-carbon wall (e.g., a wall comprising a metal, silicon, boron nitride, etc.). In some embodiments, the carbon-based wall may surround at least one non-carbon wall. In some instances, a non-carbon wall may surround at least one carbon-based wall.

The term “quantum dot” is given its normal meaning in the art and can refer to semiconducting nanoparticles that exhibit quantum confinement effects. Generally, energy (e.g., light) incident upon a quantum dot can excite the quantum dot to an excited state, after which, the quantum dot can emit energy corresponding to the energy band gap between its excited state and its ground state. Examples of materials from which quantum dots can be made include PbS, PbSe, CdS, CdSe, ZnS, and ZnSe, among others.

In some embodiments, a nanoparticle can include a coated nanoparticle. The coated nanoparticle can be a nanoparticle having an inorganic outer coating. The inorganic outer coating can include silicon or silicon dioxide. The nanoparticle can be a nanophosphor. The nanophosphor can have a peak emission wavelength of between 360 nm and 580 nm, or between 400 nm and 540 nm. The nanophosphor can have an excitation wavelength of between 200 nm and 450 nm, or between 200 nm and 450 nm. The nanophosphor can be a strontium aluminate or a zinc sulfide. The strontium aluminate can be doped with a transition metal element, a rare earth element or a lanthanide element, for example, Sc, Y, La, Ce, Pr, Nd, Pm, Sm, Eu, Gd, Tb, Dy, Ho, Er, Tm, Yb, Lu, Ti, Zr, Hf, V, Nb, Ta, Cr, Mo, W, Mn, Tc, Re, Fe, Ru, Os, Co, Rh, Ir, Ni, Pd, Pt, Cu, Ag, Su, Zn, Cd, and Hg.

A coated nanoparticle can be, in some cases, substantially free of dopants, impurities, or other non-nanoparticle atoms.

In some embodiments, a photoluminescent nanoparticle may emit radiation within a desired range of wavelengths. For example, in some cases, a photoluminescent nanoparticle may emit radiation with a wavelength between about 750 nm and about 1600 nm, or between about 900 nm and about 1400 nm (e.g., in the near-infrared range of wavelengths). In some embodiments, a photoluminescent nanoparticle may emit radiation with a wavelength within the visible range of the spectrum (e.g., between about 400 nm and about 700 nm).

In some embodiments, a coated nanoparticle may be substantially free of covalent bonds with other entities (e.g., other nanoparticles, the surface of a container, a polymer, an analyte, etc.). The absence of covalent bonding between a photoluminescent nanoparticle and another entity may, for example, preserve the photoluminescent character of the nanoparticle. In some cases, single-walled carbon nanotubes or other photoluminescent nanoparticles may exhibit modified or substantially no fluorescence upon forming a covalent bond with another entity (e.g., another nanoparticle, a current collector, a surface of a container, and the like).

A coated nanoparticle can be strongly cationic or anionic. Strongly cationic or anionic can mean that the coated nanoparticle (or other element) has a high magnitude of the zeta potential. For example, the nanoparticle can have a zeta potential of less than −10 mV or greater than 10 mV. In preferred embodiments, the nanoparticle can have a zeta potential of less than −20 mV or greater than 20 mV, a zeta potential of less than −30 mV or greater than 30 mV, or a zeta potential of less than −40 mV or greater than 40 mV.

A coated nanoparticle can include a coating or be suspended in a coating with a high magnitude of the zeta potential. A coating can be a polymer. A variety of polymers may be used in association with the embodiments described herein. In some cases, the polymer may be a polypeptide. In some embodiments, the length and/or weight of the polypeptide may fall within a specific range. For example, the polypeptide may include, in some embodiments, between about 5 and about 50, or between about 5 and about 30 amino acid residues. In some cases, the polypeptide may have a molecular weight of between about 400 g/mol and about 10,000 g/mol, or between about 400 g/mol and about 600 g/mol. Examples of protein polymers can include glucose oxidase, bovine serum albumin and alcohol dehydrogenase.

A polymer may include a linear or branched synthetic polymer (e.g., polybrene, polyethyleneimine, poly(ethylene oxide), poly(vinyl pyrrolidinone), poly(allyl amine), poly(2-vinylpyridine), and the like), in some embodiments.

A polymer may include a natural polymer, for example, histone and collagen, in some embodiments.

In some embodiments, the polymer may include an oligonucleotide. The oligonucleotide can be, in some cases, a single-stranded DNA oligonucleotide. The single-stranded DNA oligonucleotide can, in some cases, include a majority (>50%) A or T nucleobases. In some embodiments, single-stranded DNA oligonucleotide can include more than 75%, more than 80%, more than 90%, or more than 95% A or T nucleobases. In some embodiments, the single-stranded DNA oligonucleotide can include a repeat of A and T. For example, a oligonucleotide can be, in some cases, at least 5, at least 10, at least 15, between 5 and 25, between 5 and 15, or between 5 and 10 repeating units, in succession, of (GT) or (AT). Repeating units can include at least 2 nucleobases, at least 3 nucleobases, at least 4 nucleobases, at least 5 nucleotides long. The nucleobases described herein are given their standard one-letter abbreviations: cytosine (C), guanine (G), adenine (A), and thymine (T).

In some embodiments, the polymer can include a polysaccharide such as, for example, cyclodextran, chitosan, or chitin.

In some embodiments, the polymer can include an oligopeptide or a polypeptide, for example, polylysine, polyhistidine, polyornithine or polyarginine.

In preferred embodiments, the interaction between a polymer and a nanoparticle can be non-covalent (e.g., via van der Waals interactions); however, a polymer can covalently bond with a nanoparticle. In some embodiments, the polymer may be capable of participating in a pi-pi interaction with the nanostructure. A pi-pi interaction (a.k.a., “pi-pi stacking”) is a phenomenon known to those of ordinary skill in the art, and generally refers to a stacked arrangement of molecules adopted due to interatomic interactions. Pi-pi interactions can occur, for example, between two aromatic molecules. If the polymer includes relatively large groups, pi-pi interaction can be reduced or eliminated due to steric hindrance. Hence, in certain embodiments, the polymer may be selected or altered such that steric hindrance does not inhibit or prevent pi-pi interactions. One of ordinary skill in the art can determine whether a polymer is capable or participating in pi-pi interactions with a nanostructure.

The polymer complexed nanoparticles may be strongly cationic or anionic, meaning that the polymer has a high magnitude of the zeta potential. For example, the polymer can have a zeta potential of less than −10 mV or greater than 10 mV, less than −20 mV or greater than 20 mV, less than −30 mV or greater than 30 mV, or less than −40 mV or greater than 40 mV.

A nanoparticle can be contained within a chloroplast, as demonstrated more fully herein. A nanoparticle can traverse and/or localize within the outer membrane layer (i.e., lipid bilayer). The process can be complete and/or irreversible. Because other organelles include an outer membrane layer (i.e., lipid bilayer), a nanoparticle can be contained within other organelles. For example, other organelles that a nanoparticle can be introduced into can include a nucleus, endoplasmic reticulum, Golgi apparatus, chloroplast, chromoplast, gerontoplast, leucoplast, lysosome, peroxisome, glyoxysome, endosome, mitochondria or vacuole.

Thylakoids are a membrane-bound compartment inside a chloroplast. Cyanobacteria can also include thylakoids. In some embodiments, a nanoparticle can be associated with a thylakoid membrane within a chloroplast, cyanobacteria or other photocatalytic cell or organelle.

A nanoparticle can be contained within a photocatalytic unit, most preferably, including an outer lipid membrane (i.e., lipid bilayer). A photocatalytic unit can be a structure capable of performing photosynthesis or photocatalysis, preferably a cell or an organelle capable of performing photosynthesis or photocatalysis. For example, a photocatalytic unit can be a chloroplast, a cyanobacteria, or a bacterial species selected from the group consisting of Chlorobiacea spp., a Chromaticacea spp. and a Rhodospirillacae spp.

An organelle can be part of a cell, a cell can be part of a tissue, and a tissue can be part of an organism. For example, a nanoparticle can be contained within a cell of a leaf of a plant. More to the point, a cell can be intact. In other words, the organelle may not be an isolated organelle, but rather, the organelle can be contained within the outer lipid membrane of a cell.

A nanoparticle that is independent of an organelle or cell can be free of lipids. An outer lipid membrane can enclose or encompass an organelle or cell. As the nanoparticle traverses the outer lipid membrane of an organelle or cell, lipids from the outer lipid membrane can associate or coat the nanoparticle. As a result, a nanoparticle inside the outer lipid membrane of an organelle or cell can be associated with or coated with lipids that originated in the organelle or cell.

Transport of a nanoparticle into an organelle or a cell can be a passive process. In some cases, transport across the outer lipid membrane can be independent of the temperature or light conditions.

Embedding a nanoparticle within an organelle or cell can be useful for monitoring the activity of the organelle or cell. For example, a nanoparticle, preferably a photoluminescent nanoparticle, can be introduced into the organelle or cell. Measurements of the photoluminescence of a photoluminescent nanoparticle can provide information regarding a stimulus within an organelle or cell. Measurements of the photoluminescence of a photoluminescent nanoparticle can be taken at a plurality of time points. A change in the photoluminescence emission between a first time point and a second time point can indicate a change in a stimulus within the organelle or cell.

In some embodiments, a change in the photoluminescence emission can include a change in the photoluminescence intensity, a change in an emission peak width, a change in an emission peak wavelength, a Raman shift, or combination thereof. One of ordinary skill in the art would be capable of calculating the overall intensity by, for example, taking the sum of the intensities of the emissions over a range of wavelengths emitted by a nanoparticle. In some cases, a nanoparticle may have a first overall intensity, and a second, lower overall intensity when a stimulus changes within the organelle or cell. In some cases, a nanoparticle may emit a first emission of a first overall intensity, and a second emission of a second overall intensity that is different from the first overall intensity (e.g., larger, smaller) when a stimulus changes within the organelle or cell.

A nanoparticle may, in some cases, emit an emission of radiation with one or more distinguishable peaks. One of ordinary skill in the art would understand a peak to refer to a local maximum in the intensity of the electromagnetic radiation, for example, when viewed as a plot of intensity as a function of wavelength. In some embodiments, a nanoparticle may emit electromagnetic radiation with a specific set of peaks. In some cases, a change in a stimulus may cause the nanoparticle to emit electromagnetic radiation including one or more peaks such that the peaks (e.g., the frequencies of the peaks, the intensity of the peaks) may be distinguishable from one or more peaks prior to the change in stimulus. In some cases, the change in a stimulus may cause the nanoparticle to emit electromagnetic radiation comprising one or more peaks such that peaks (e.g., the frequencies of the peaks, the intensity of the peaks) are distinguishable from the one or more peaks observed prior to the change in the stimulus. When the stimulus is the concentration of an analyte, the frequencies and/or intensities of the peaks may, in some instances, allow one to determine the analyte interacting with the nanoparticle by, for example, producing a signature that is unique to a particular analyte that is interacting with the nanoparticle. Determination of a specific analyte can be accomplished, for example, by comparing the properties of the peaks emitted in the presence of the analyte to a set of data (e.g., a library of peak data for a predetermined list of analytes).

A stimulus can include the pH of the organelle or cell. A change in the pH can be an increase or decrease in the pH.

A stimulus can include a modification of an analyte. For example, an analyte may be oxidized or reduced. In other examples, an analyte can be ionized. In another example, an analyte can include an ether, ester, acyl, or disulfide or other derivative.

A stimulus can include the concentration of an analyte. An analyte can include a reactive oxygen species, for example, hydrogen peroxide, superoxide, nitric oxide, and a peroxidase. Alternatively, an analyte can be carbon dioxide, adenosine triphosphate (ATP), nicotinamide adenine dinucleotide phosphate (NADP⁺ or NADPH), or oxygen. In some instances, the concentration of the analyte may be relatively low (e.g., less than about 100 micromolar, less than about 10 micromolar, less than about 1 micromolar, less than about 100 nanomolar, less than about 10 nanomolar, less than about 1 nanomolar, or about a single molecule of the analyte). In some cases, the concentration of an analyte may be zero, indicating that no analyte is present.

Chloroplasts can be considered a high source of chemical energy in food supplies and carbon-based fuels on the planet. By capturing atmospheric CO₂, these plant organelles convert light energy into three major forms of sugars that fuel plant growth: maltose, triose phosphate and glucose. (Weise, S. E., Weber, A. P. M. & Sharkey, T. D. Maltose is the major form of carbon exported from the chloroplast at night. Planta 218, 474-82 (2004), which is incorporated by reference in its entirety). While some information exists on the interface between photosystems and nanomaterials, nanoengineering chloroplast photosynthesis for enhancing solar energy harnessing remains unexplored. (Boghossian, A. A. et al. Application of Nanoparticle Antioxidants to Enable Hyperstable Chloroplasts for Solar Energy Harvesting. Adv. Energy Mater. 3:7, p. 881-893 (2013), which is incorporated by reference in its entirety). One deterrent in using chloroplast photosynthetic power as an alternative energy source can be that these organelles are no longer independently living organisms. However, isolated chloroplasts from the algae Vaucheria litorea in symbiotic association with the sea slug Elysia chlorotica remarkably can remain functional for at least 9 months. (Trench, R. K., Boyle, J. E. & Smith, D. C. The Association between Chloroplasts of Codium fragile and the Mollusc Elysia viridis. I. Characteristics of Isolated Codium Chloroplasts. Proc. R. Soc. B Biol. Sci. 184, 51-61 (1973); and Rumpho, M. E., Summer, E. J. & Manhart, J. R. Solar-Powered Sea Slugs. Mollusc/Algal Chloroplast Symbiosis. 123, 29-38 (2000), each of which is incorporated by reference in its entirety). Land plant chloroplast photosystem activity can decline within a day after extraction, while ex vivo sugar output can last for only a few hours. (Weise, S. E., et al. (2004); Choe, H. & Thimann, K. The Senescence of Isolated Chloroplasts. Planta 121, 201-203 (1974); Green, B. J., Fox, T. C. & Rumpho, M. E. Stability of isolated algal chloroplasts that participate in a unique mollus/kleptoplast association. Symbiosis 40, 31-40 (2005); and Neuhaus, H. E. & Schulte, N. Starch degradation in chloroplasts isolated from C3 or CAM (crassulacean acid metabolism)-induced Mesembryanthemum crystallinum L. Biochem. J. 318, 945-53 (1996), each of which is incorporated by reference in its entirety). Although chloroplasts have mechanisms in place to self-repair photo-damaged proteins, a double-stranded circular DNA with a subset of protein-encoding genes involved in photosynthesis, and ribosomal units for protein synthesis and assembly, little is known about engineering these plant organelles for long-term, stable photosynthesis ex vivo. (Edelman, M. & Mattoo, A. K. D1-protein dynamics in photosystem II: the lingering enigma. Photosynth. Res. 98, 609-20 (2008); Schmitz-Linneweber, C. et al. The plastid chromosome of spinach (Spinacia oleracea): complete nucleotide sequence and gene organization. Plant Mol. Biol. 45, 307-15 (2001); and Marin-Navarro, J., Manuell, A. L., Wu, J. & P Mayfield, S. Chloroplast translation regulation. Photosynth. Res. 94, 359-74 (2007), each of which is incorporated by reference in its entirety). Another limitation of chloroplasts photosynthesis can be that absorbed light is constrained to the visible range of the spectrum, allowing access to only roughly 50% of the incident solar energy radiation. (Bolton, J. R. & Hall, D. Photochemical conversion and storage of solar energy. Annu. Rev. Energy 4, 353-401 (1979), which is incorporated by reference in its entirety). Furthermore, in some conditions, less than 10% of full sunlight saturates the capacity of the photosynthetic apparatus. (Zhu, X.-G., Long, S. P. & Ort, D. R. Improving photosynthetic efficiency for greater yield. Annu. Rev. Plant Biol. 61, 235-61 (2010), which is incorporated by reference in its entirety). Photosynthetic organisms appear to have evolved for reproductive success, including shading competitors, not for solar energy conversion efficiency. Thus, improving photosynthetic efficiency may require extending the range of solar light absorption, particularly in the near infrared spectral range, which is able to penetrate deeper into living organisms. (Blankenship, R. E. et al. Comparing photosynthetic and photovoltaic efficiencies and recognizing the potential for improvement. Science 332, 805-9 (2011), which is incorporated by reference in its entirety).

The high stability and unique chemical and physical traits of nanomaterials have the potential to enable chloroplast-based photocatalytic complexes both ex vivo and in vivo with enhanced and novel functional properties. Single-walled carbon nanotubes (“SWNTs”) embedded within chloroplasts have the potential to enhance the light reactions of photosynthesis with their distinctive optical and electronic properties. Under bright sunlight, chloroplast photosystems can capture more photons than they can convert into electron flow. (Wilhelm, C. & Selmar, D. Energy dissipation is an essential mechanism to sustain the viability of plants: The physiological limits of improved photosynthesis. J. Plant Physiol. 168, 79-87 (2011), which is incorporated by reference in its entirety). However, under non-saturating light conditions, maximizing solar energy capture can be crucial. (Scholes, G. D., Fleming, G. R., Olaya-Castro, A. & van Grondelle, R. Lessons from nature about solar light harvesting. Nat. Chem. 3, 763-774 (2011), which is incorporated by reference in its entirety). SWNTs can absorb light over a broad range of wavelengths in the ultraviolet, visible and nIR spectra not captured by the chloroplast antenna pigments. The electronic band gap of semiconducting SWNTs can allow them to convert this absorbed solar energy into excitons that could transfer electrons to the photosynthetic machinery. (Han, J.-H. et al. Exciton antennas and concentrators from core-shell and corrugated carbon nanotube filaments of homogeneous composition. Nat. Mater. 9, 833-9 (2010), which is incorporated by reference in its entirety). Also, SWNT-based nanosensors can monitor single-molecule dynamics of free radicals within chloroplasts for optimizing photosynthetic environmental conditions (light and CO₂). (Zhang, J. et al. Single Molecule Detection of Nitric Oxide Enabled by d(AT)(15) DNA Adsorbed to Near Infrared Fluorescent Single-Walled Carbon Nanotubes. J. Am. Chem. Soc. 20, 567-581 (2010), which is incorporated by reference in its entirety). However, nanoengineering photosynthesis can require the delivery of nanomaterials through the chloroplast outer envelope. Nanoparticle transport through lipid bilayers has been described to be energy dependent, requiring endocytosis pathways that have not been reported in isolated chloroplasts. (Shi, X., von dem Bussche, A., Hurt, R. H., Kane, A. B. & Gao, H. Cell entry of one-dimensional nanomaterials occurs by tip recognition and rotation. Nat. Nanotechnol. 6, 714-9 (2011), which is incorporated by reference in its entirety). To date, nanomaterial uptake mechanisms through cell membranes are controversial and poorly understood in organelles like chloroplasts. (Pogodin, S., Slater, N. K. H. & Baulin, V. a. Surface patterning of carbon nanotubes can enhance their penetration through a phospholipid bilayer. ACS Nano 5, 1141-6 (2011), which is incorporated by reference in its entirety).

The interface between plant organelles and non-biological nanoparticles has the potential to impart the former with new and enhanced functions. For example, this nanobionic approach can yield chloroplasts that possess enhanced photosynthetic activity both ex vivo and in vivo, are more stable to reactive oxygen species ex vivo, and allow real time information exchange via embedded nanosensors for free radicals in plants. Accordingly, there is a need for nanoparticles that can interface with organelles, specifically, plant organelles ex vivo and in vivo to enable novel or enhanced functions. Similarly, there is a need for nanoparticles that can interface with intact photosynthetic organisms or intact cells of photosynthetic organisms ex vivo and in vivo to enable novel or enhanced functions. For example, the assembly of nanoparticle complexes within chloroplast photosynthetic machinery has the potential to enhance solar energy conversion through augmented light reactions of photosynthesis and ROS scavenging while imparting novel sensing capabilities to living plants.

Phosphor materials provide phosphorescence for several hours after excitation, which carries interest for applications in many light active devices. However, the size-dependent phosphorescence of colloidal nanoparticles such as strontium aluminate (SrAl204:Eu²⁺, Dy³⁺) has not yet been studied, due to a weakened ability to control size caused by high thermal synthetic strategies and poor particle solubility. Herein, wet milled and then ultra-sonicated pristine nanoparticles of SrAl204:Eu²⁺, Dy³⁺ result in particle diameters ranging from 481.5±26.0 to 51.9±6.4 nm in colloidally stable solutions. In addition, milled strontium aluminate particles were Si/SiO₂ coated and sorted by centrifuging to discrete, final diameters ranging from 262.0±24.2 nm to 384.5±48.7 nm. Photophysical properties investigated across sizes were compared for both set of samples. Gradual changes in photoluminescence emission from 532 nm to 508 nm (˜24 nm) were observed under pristine particle milling, clearly indicating the size dependence. In contrast, Si/SiO₂ coated nanophosphors show the opposite (red) shift in emission, with a new peak intensity at 555 nm (˜53 nm), about ˜23 nm more than raw material. Both sets of materials demonstrated a longer decay time per volume (˜22% for milled particles and ˜10% for Si/SiO₂ modified nanoparticles) upon particle size reduction, demonstrating the advantage of this nanomaterial.

Phosphor materials have unique persistent afterglow properties, important for use in fluorescent lamps, electroluminescent, numerical and graphical displays², light emitting diodes and glowing polymer composites. See, for example, Yu, Y., Wang, J., Zhu, Y. N. & Ge, M. Q. Researches on preparation and properties of polypropylene nonwovens containing rare earth luminous materials. J Rare Earth 32, 1196-1200, doi:10.1016/S1002-0721(14)60203-9 (2014), Guo, Y. T. & Huang, Y. M. Green aluminate phosphors used for information display. Key Eng Mat 428-429, 421-425, doi:10.4028/(2010), Jamalaiah, B. C. & Babu, Y. R. Near UV excited SrAl204:Dy3+ phosphors for white LED applications. Mater Chem Phys 211, 181-191, doi:10.1016/j.matchemphys.2018.02.025 (2018), Van der Heggen, D., Joos, J. J. & Smet, P. F. Importance of Evaluating the Intensity Dependency of the Quantum Efficiency: Impact on LEDs and Persistent Phosphors. Acs Photonics 5, 4529-4537, doi:10.1021/acsphotonics.8b00979 (2018), Li, Y., Gecevicius, M. & Qiu, J. R. Long persistent phosphors—from fundamentals to applications. Chem Soc Rev 45, 2090-2136, doi:10.1039/c5cs00582e (2016), and Do, Y. R. & Bae, J. W. Application of photoluminescence phosphors to a phosphor-liquid crystal display. J Appl Phys 88, 4660-4665, doi:Doi 10.1063/1.1311825 (2000), each of which is incorporated by reference in its entirety. Despite the large demand, technologies utilizing phosphor materials still lack solution methodology involving stable nanomaterial dispersions that are compatible with fast and cheap processing techniques such as spin coating, spraying or printing. The main limiting factors are related to their low chemical stability, fast sedimentation in solvents and lack of size-controlled fabrication methods. The relationship between particle size, photoluminescence (PL) and lifetime (i.e., afterglow property), which strongly depends on synthetic strategy and surface modifications, are not well studied to date. Therefore, its continued study is of great interest for applications in nanoscience, materials science and biology. To approach this challenge, size-sorting strategies were developed for collecting pristine and Si/SiO₂ coated phosphor nanoparticles.

The first persistent phosphorescence was discovered by Matsuzawa in strontium aluminate oxide doped with the rare Earth elements Europium (Eu) and Dysprosium (Dy) (SrAl2O₄:Eu²⁺, Dy³⁺) prepared by high thermal treatments. These ions support the afterglow mechanism, which is realized by the slow thermal release of trapped holes at Dy³⁺ ions states embedded in the SrAl₂O₄ matrix, while the excited electrons during the 4f→5d excitation remain captured at Eu⁺² ions (phosphor charging process under UV illumination). This mechanism is discussed in more detail previously. See, for example, Matsuzawa, T., Aoki, Y., Takeuchi, N. & Murayama, Y. New long phosphorescent phosphor with high brightness, SrAl₂O₄:Eu²⁺,Dy³⁺. J Electrochem Soc 143, 2670-2673, doi:Doi 10.1149/1.1837067 (1996), Takasaki, H., Tanabe, S. & Hanada, T. Long-lasting afterglow characteristics of Eu, Dy codoped SrO—Al₂O₃ phosphor. J Ceram Soc Jpn 104, 322-326, doi:10.2109/jcersj.104.322 (1996), and Yamamoto, H. & Matsuzawa, T. Mechanism of long phosphorescence of SrAl₂O₄:Eu2+, Dy3+ and CaAl₂O₄:Eu2+, Nd3+. J Lumin 72-4, 287-289, doi:Doi 10.1016/50022-2313(97)00012-4 (1997), each of which is incorporated by reference in its entirety. Different synthetic routes such as sol-gel method, hydrothermal synthesis, chemical precipitation, and solid-state synthetic methods that lead to fabrication of various phosphor particles of different compositions and sizes were developed, however, strontium aluminate maintains the superior choice because of its prolonged afterglow time. See, for example, Li, Y., Gecevicius, M. & Qiu, J. R. Long persistent phosphors—from fundamentals to applications. Chem Soc Rev 45, 2090-2136, doi:10.1039/c5cs00582e (2016), Peng, T. Y., Huajun, L., Yang, H. P. & Yan, C. H. Synthesis of SrAl2O4: Eu, Dy phosphor nanometer powders by sol-gel processes and its optical properties. Mater Chem Phys 85, 68-72, doi:10.1016/j.matchemphys.2003.12.001 (2004), Julian-Lopez, B. et al. Self-assembling of Er(2)O(3)-TiO(2) mixed oxide nanoplatelets by a template-free solvothermal route. Chemistry 15, 12426-12434, doi:10.1002/chem.200901423 (2009), Tao, J. H. et al. Controls of Tricalcium Phosphate Single-Crystal Formation from Its Amorphous Precursor by Interfacial Energy. Cryst Growth Des 9, 3154-3160, doi:10.1021/cg801130w (2009), Zhang, R. X., Han, G. Y., Zhang, L. W. & Yang, B. S. Gel combustion synthesis and luminescence properties of nanoparticles of monoclinic SrAl₂O₄:Eu²⁺,Dy³⁺. Mater Chem Phys 113, 255-259, doi:10.1016/j.matchemphys.2008.07.084 (2009), Rezende, M. V. D., Montes, P. J. R., Soares, F. M. D., dos Santos, C. & Valerio, M. E. G. Influence of co-dopant in the europium reduction in SrAl204 host. J Synchrotron Radiat 21, 143-148, doi:10.1107/1600577513025708 (2014), Suriyamurthy, N. & Panigrahi, B. S. Effects of non-stoichiometry and substitution on photoluminescence and afterglow luminescence of Sr₄Al₁₄O₂₅:Eu²⁺,Dy³⁺ phosphor. J Lumin 128, 1809-1814, doi:10.1016/j.jlumin.2008.05.001 (2008), He, Z. Y., Wang, X. J. & Yen, W. M. Investigation on charging processes and phosphorescent efficiency of SrAl₂O₄:Eu,Dy. J Lumin 119, 309-313, doi:10.1016/j.jlumin.2006.01.010 (2006), each of which is incorporated by reference in its entirety.

Fabricating phosphor material by a solid-state reaction requires thermal treatment at 1300-1900° C., which results in a large powder containing particles 100 μm in diameter. In contrast, the developed combustion approach is more suitable for fabrication of nanoparticles because it requires a reduced (1100° C.) temperature and ambient conditions suitable for particles fabrication of different compositions. See, for example, Qiu, Z. F., Zhou, Y. Y., Lu, M. K., Zhang, A. Y. & Ma, Q. Combustion synthesis of three-dimensional reticular-structured luminescence SrAl₂O₄: Eu,Dy nanocrystals. Solid State Sci 10, 629-633, doi:10.1016/j.solidstatesciences.2007.10.009 (2008), which is incorporated by reference in its entirety. Nevertheless, the combustion strategy still lacks the ability to effectively size control particles within different ranges. It was also demonstrated that fabricated SrAl₂O₄:Eu²⁺,Dy³⁺ nanoparticles of 40-50 nm have a blue shift in emission wavelength from 530 nm to 515 nm. See, for example, Yu, X. B., Zhou, C. L., He, X. H., Peng, Z. F. & Yang, S. P. The influence of some processing conditions on luminescence of SrAl₂O₄:Eu²⁺ nanoparticles produced by combustion method. Mater Lett 58, 1087-1091, doi:10.1016/j.matlet.2003.08.022 (2004), which is incorporated by reference in its entirety. Similarly, the utilized microwave route for SrAl₂O₄:Eu²⁺,Dy³⁺ particles ranging in size from 4.8 μm to 14.4 μm leads to a characteristic blue shift from 518 nm to 507 nm respectively. See, for example, Geng, J. & Wu, Z. Synthesis of long afterglow SrAl₂O₄:Eu²⁺, Dy³⁺ phosphors through microwave route. J Mater Synth Proces 10, 245-248, doi:Doi 10.1023/A:1023038008386 (2002), which is incorporated by reference in its entirety. Observed changes in the post glowing emission under particle size reduction in the powder material were linked to the possible effects happening on their surface. For example, the creation of dipper energy levels leading to the emission of blue light or the building up of additional crystal field strength around Eu²⁺ ions, which modify their energetic states, however, not studied in details. Similarly, SrAl₂O₄:Eu²⁺, Dy³⁺ nanoparticles of 3170 nm and 26 nm prepared by the sol-gel method showed a blue shift from 520 nm to 500 nm for smaller nanoparticles respectively, motivated by the presence of chemisorbed species on particle surfaces leading charge transfer process. See, for example, Tang, Z. L., Zhang, F., Zhang, Z. T., Huang, C. Y. & Lin, Y. H. Luminescent properties of SrAl₂O₄: Eu, Dy material prepared by the gel method. J Eur Ceram Soc 20, 2129-2132, doi:Doi 10.1016/50955-2219(00)00092-3 (2000), which is incorporated by reference in its entirety. At the same time, the emission intensity of smaller particles of the same volume was reduced, similarly, a detailed study of observed changes and corresponding effect their colloidal solutions were not realized.

In contrast, the gel synthetic method applied to Sr₂ZnSi₂O₇:Eu²⁺, Dy³⁺ nanophosphor synthesis, producing particles of diameter 49, 56 and 62 nm, showed that smaller nanoparticles have a brighter emission spectra and much longer decay time in comparison to the same bulk material, which is due to an enlarged number of defects in fabricated nanoparticles in contrast to the bulk material. See, for example, Wang, X. J. et al. Crystal size dependence of the persistent phosphorescence in Sr₂ZnSi₂O₇:Eu²⁺,Dy³⁺. Microelectron J 36, 546-548, doi:10.1016/j.mejo.2005.02.067 (2005), which is incorporated by reference in its entirety. However, no changes in the emission spectrum were detected. Similarly, 1.0 μm, 0.1 μm and 0.05 μm particles of Sr₄Al₄O₂₅:Eu²⁺/Dy³⁺ fabricated by modified solid method synthesis resulted in the gradual increase in the emission spectrum, respectively, upon particle size reduction, supporting the hypothesis that smaller nanomaterials have a brighter emission intensity with no changes in the emission wavelength. See, for example, Hom Nath Luitel, T. W., Toshio Torikai, Mitsunori Yada. Effects of Particle Size and Type of Alumina on the Morphology and Photoluminescence Properties of Sr₄Al₁₄O₂₅:Eu²⁺/Dy³⁺ Phosphor. Research Letters in Materials Science (2009), which is incorporated by reference in its entirety.

An alternative to the synthetic methods described above is the wet milling strategy, which begins with large phosphor nanoparticles and over a duration of several days results in the reduction of particle size with no changes in the emission spectrum. See, for example, Rojas-Hernandez, R. E., Rubio-Marcos, F., Enriquez, E., De La Rubia, M. A. & Fernandez, J. F. A low-energy milling approach to reduce particle size maintains the luminescence of strontium aluminates. Rsc Adv 5, 42559-42567, doi:10.1039/c5ra04878h (2015), which is incorporated by reference in its entirety. Afterwards, the particles can be stabilized further with an additional Si/SiO₂ shell or polyethylene glycol coating. See, for example, Paterson, A. S. et al. Persistent Luminescence Strontium Aluminate Nanoparticles as Reporters in Lateral Flow Assays. Anal Chem 86, 9481-9488, doi:10.1021/ac5012624 (2014) and Sun, M. et al. Persistent luminescent nanoparticles for super-long time in vivo and in situ imaging with repeatable excitation. J Lumin 145, 838-842, doi:10.1016/j.jlumin.2013.08.070 (2014), each of which is incorporated by reference in its entirety. Applied encapsulation strategies such as these are important for particle stability and for applications in devices or in vivo imaging strategies, since some types of non-modified phosphorescent material have limited stability in aqueous solutions and therefore undergo fast denaturation and a rapid loss of afterglow phosphorescent properties. See, for example, Zhu, Y., Zheng, M. T., Zeng, J. H., Xiao, Y. & Liu, Y. L. Luminescence enhancing encapsulation for strontium aluminate phosphors with phosphate. Mater Chem Phys 113, 721-726, doi:10.1016/j.matchemphys.2008.08.007 (2009), which is incorporated by reference in its entirety. For example, non-coated Sr₄Al₄O₂₅:Eu²⁺, Dy³⁺ prepared by the chemical vapor deposition method only showed activity for three days after being exposed to serum conditions. See, for example, Chen, H. M. et al. Nanoscintillator-Mediated X-ray Inducible Photodynamic Therapy for In Vivo Cancer Treatment. Nano Lett 15, 2249-2256, doi:10.1021/nl504044p (2015), which is incorporated by reference in its entirety.

In this work, the afterglow mechanism of colloidal stable phosphor nanoparticles of different sizes were investigated, both non-modified and modified with a Si/SiO₂ shell, the latter of which resulted in better particle stability and solubility in water. Two strategies for particle size control have been developed, such as ultrasonication as particles post milling and the integrated centrifuging method for Si/SiO₂ coated analogs, which resulted in the smallest sized nanoparticles (sub-100 nm in diameter). Size-dependent changes in afterglow emission light are reported here for the first time, in the form of shifts in both blue and red directions. The nanomaterials described here have potential for applications in the fabrication of functional materials, biologic sensing platforms, afterglow printer inks and time gated detection devices.

### Experimental Methods

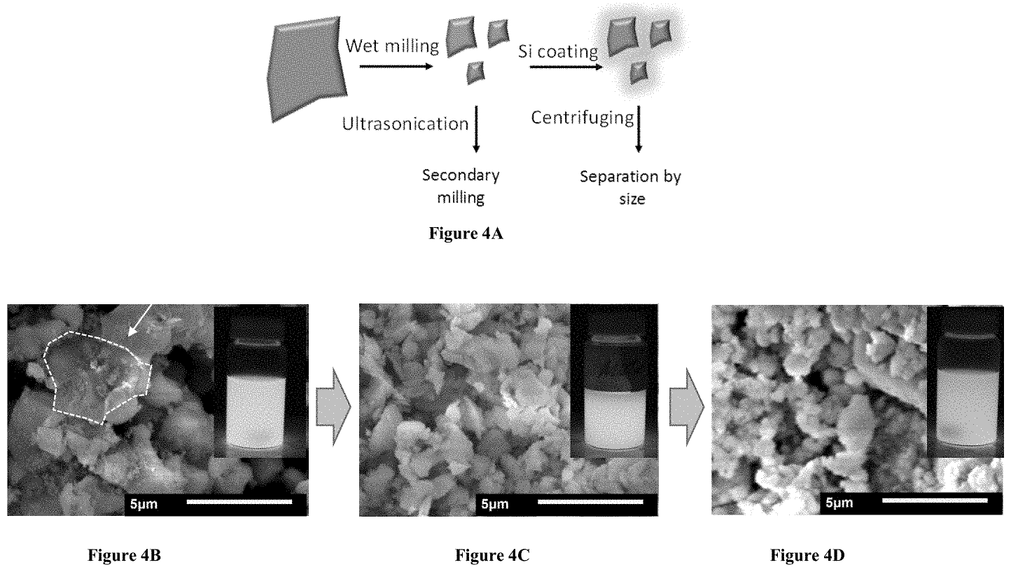

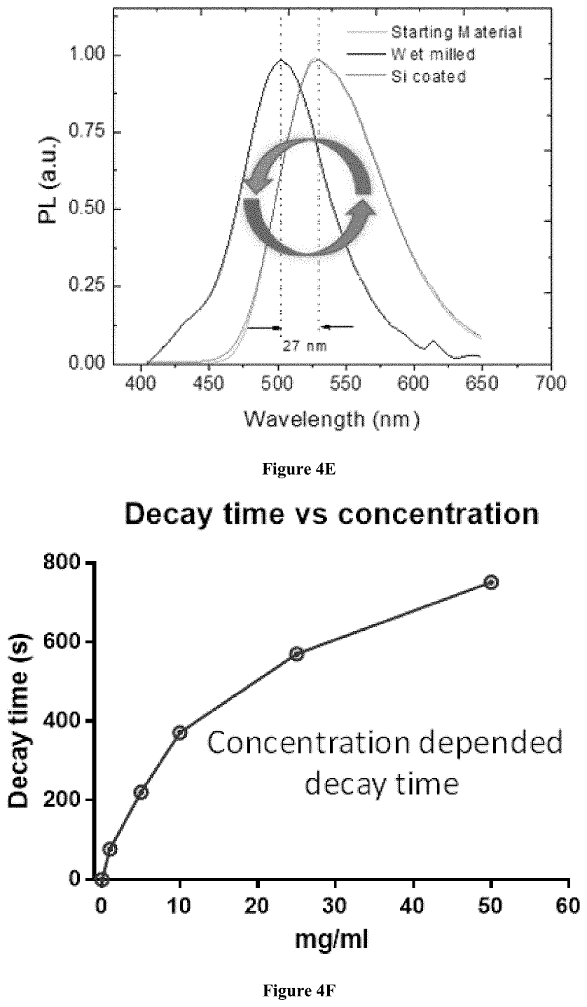

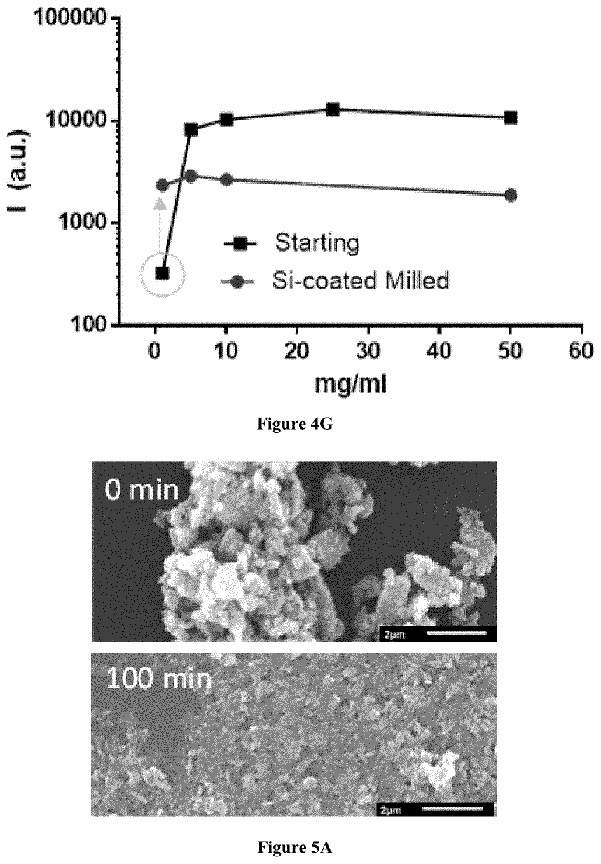

Commercially available strontium aluminate powder with an average particle size of 3 μm, as analyzed by transmission electron microscopy (TEM), was wet milled (see Methods) for 7-14 days in ethyl acetate. Next, the milled sample was used for additional Si/SiO₂ coating to provide better sample solubility and stability. Final samples of raw (starting) material, wet milled and wet milled sample with Si/SiO₂ coating were studied for both PL and time dependent emission experiments. Because the milling procedure produces smaller particles of non-uniform shapes and dimensions, the samples were treated as a poly dispersed material, which contains a broad range of particle diameters. Therefore, the impact of the milling and Si/SiO₂ coating steps themselves was studied on emission when compared to the starting material. In the following part of our work, milled and Si/SiO₂ modified particles were categorized by size with the help of (i) the ultrasonic post-milling strategy or (ii) the accumulative centrifuging for Si-coated phosphor strategy respectively, FIG. 4A.

Measurements of the photoluminescence (PL) spectra (under UV 365 nm excitation) of non-sorted pristine (analyzed by scanning electron microscopy (SEM), FIG. 4C) and wet-milled strontium aluminate of 481.526.0 nm particles (FIG. 4D) were performed on powder samples dissolved in water. A striking blue shift of 19 nm from 527.2±3.4 nm to 508.317.5 nm was observed for the milled sample, while the starting material stirred in water had PL characteristic to solid powder (FIG. 4B). In contrast, an applied Si/SiO₂ coating to the milled material revealed an additional PL shift in the opposite direction (21.9 nm) with the new emission peak position at 530.25.8 nm (FIG. 4B). Moreover, milled particles showed 52% less PL intensity than the starting sample, while the Si/SiO₂ coated particles revealed only 16% less intensity (i.e., 36% of PL was recovered by Si/SiO₂ modification) at 50 mg/ml concentrations.

Besides observed changes in PL, the applied milling step results in shorter particle afterglow time as determined by time decay constants, which was reported previously. See, for example, Paterson, A. S. et al. Persistent Luminescence Strontium Aluminate Nanoparticles as Reporters in Lateral Flow Assays. Anal Chem 86, 9481-9488, doi:10.1021/ac5012624 (2014), which is incorporated by reference in its entirety. For these experiments, the sample powder or the same amount by mass was dissolved in deionized water and kept the solution under continuous stirring in the dark for the entire time of measurement. Next, our samples were UV illuminated for 10 min (“light charging”) and subsequently a series of images were captured with a camera under 30 s exposure time with one min time interval. Measured decay time (r) for both starting material and milled revealed 751.824.6 s and 145.8±4.8 s respectively, as calculated by fitting the equation I(t)=I₀exp(−t/τ) to the decay curves, where Jo represents the afterglow intensity at zero time. Moreover, measured decay time for the series of raw materials with concentrations of 1, 5, 10, 25, and 50 mg/ml showed that the r of the more concentrated samples approaches a saturation limit, FIG. 4F. This observation confirms that phosphor particles have a non-linear relationship between mass and post excitation emission intensity, which can be partially explained by the pronounced self-shielding or self-scattering effect known as Mie and Rayleigh light scatterings, which results in sample turbidity characteristic to colloidal solutions. See, for example, Palkar, S. A., Ryde, N. P., Schure, M. R., Gupta, N. & Cole, J. B. Finite difference time domain computation of light scattering by multiple colloidal particles. Langmuir 14, 3484-3492, doi:DOI 10.1021/la971057a (1998), which is incorporated by reference in its entirety. Therefore, emitted light from highly concentrated samples will undergo multiple elastic scattering incidents, resulting in a brightness reduction before coming out. Therefore, how varying particle charging time effects decay time was also investigated.

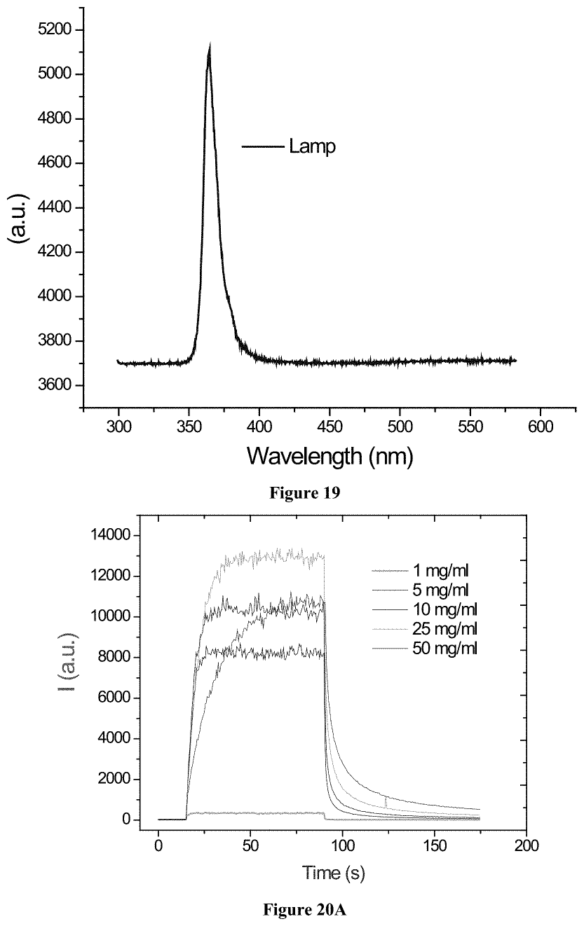

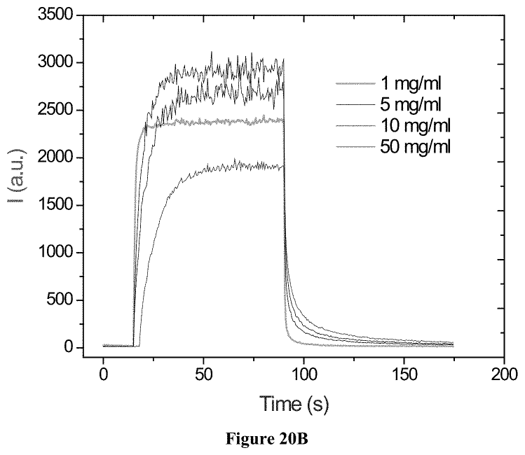

To study this, three samples of starting material, each at a concentration of 50 mg/ml, were excited with UV light for 10 min, 30 min and 60 min respectively. The measured decay time constant r showed similar values of 969.8±39.6 s, 908.4±38.76 s and 840.031.9 s, indicating that this process occurs in fast timescales with similar saturation intensities, FIG. 18. Therefore, kinetics have been investigated in more details; for this, different concentrations of materials were placed in stirred vials in front of the spectrophotometer and measured PL rise time (tᵣ) with the saturation intensity (I₀) as a function of time. For this experiment, observed rise time and slow afterglow decay was realized while the samples were shortly illuminated for 3 min with LED (spectra, FIG. 19) from the side. Then, PL spectra were recorded continuously over the duration of 10 min of the entire experiment (500 ms exposure), FIG. 20. Raw material samples were prepared with the sample concentrations of 1, 5, 10, 25, and 50 mg/ml and measured them, which clearly showed a striking difference. It was observed that the rise time is highly dependent on concentration, where measured I₀ is higher for a 25 mg/ml concentrated particle sample (I₀=12955.9±23.5 a.u) than that of the 50 mg/ml sample (I₀=10759.9±34.5 a.u), calculated by using the equation I(t)=I₀·(1−exp(−t/tᵣ)), FIG. 4G. Moreover, samples of lower concentration, such as 1 mg/ml, undergo saturation intensity much faster (tᵣ=0.89±0.07 s) than the 50 mg/ml sample (tᵣ=15.167±0.183 s). At the same time, the experiments performed on Si/SiO₂ coated milled particles of 1 mg/ml concentration showed a higher PL intensity (I₀=2348.0±6.6 a.u) than the analogical 50 mg/ml sample (I₀=1892.8±3.2 a.u.) and, again, the shortest charging time of tᵣ=0.97±0.05 s (as compared to tᵣ=8.32±0.08 s). Moreover, the Si/SiO₂ coated sample showed over a seven fold increase in I₀ than in the non-milled sample (I₀=325.8±1.4 a.u.), with the comparable rise time tᵣ values equal to 0.97±0.05 s and 0.900.08 s respectively. This sample showed clear advantages of milled particles over non-milled, demonstrating that high I₀ can be explained by reduced light scattering in the sample of smaller particles. Next, the overall strontium aluminate brightness depends not only on the particle concentration but also on their sizes.

In order to understand the role of particle size on the PL and afterglow properties, samples of different particle sizes were prepared of both milled and Si/SiO₂ coated variants by developing two size-control strategies. In contrast to Si-coated particles, which are polar and suitable for forming partially stable dispersions in solvents, milled non-modified particles show very pure colloidal stability in water by undergoing fast sedimentation into a white powder over period of 10 min. Therefore, two separate strategies of extraction of non-sedimenting nanoparticles were developed. One was through utilizing the ultrasonic method as a secondary post milling strategy for non-modified samples, and another was focused on centrifuging strategy for Si/SiO₂ coated particles sorting.

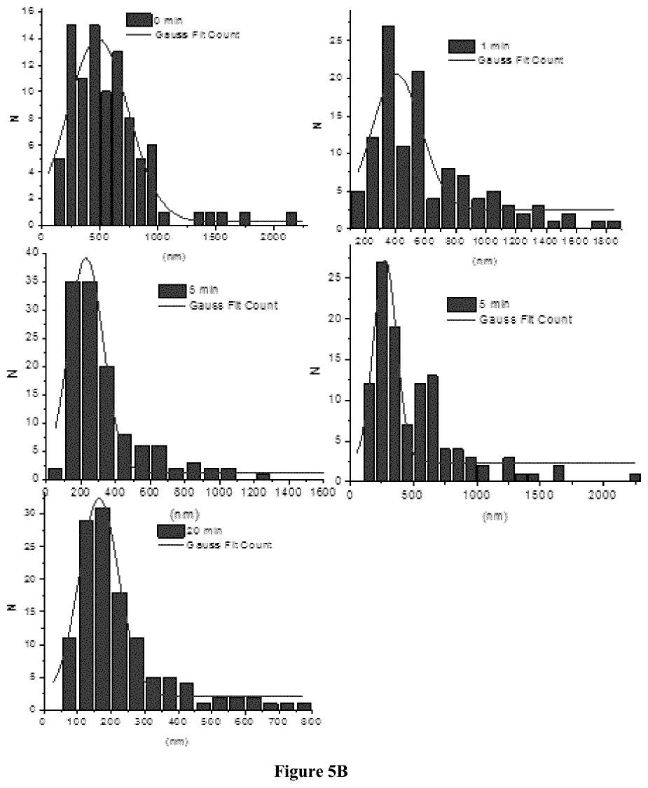

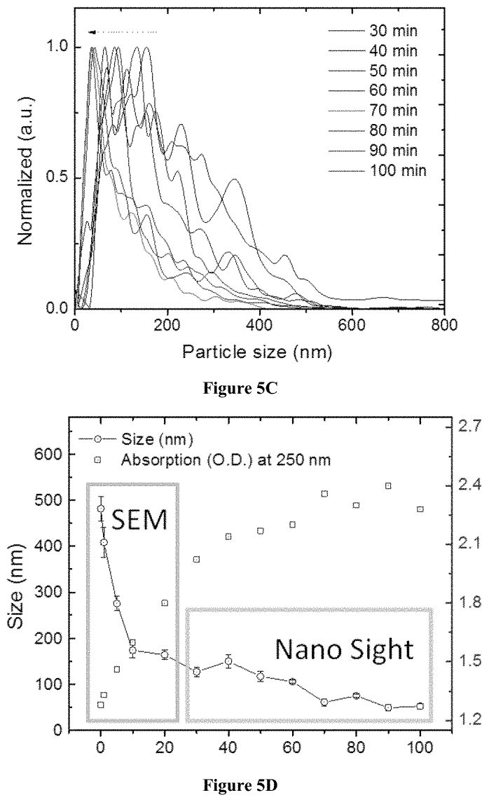

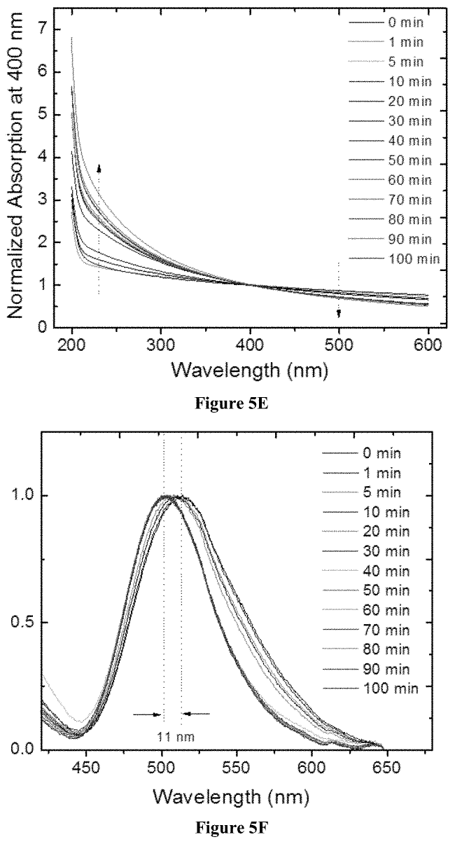

Firstly, it was observed that the applied ultrasonic method for wet-milled nanoparticles revealed drastic changes in nanoparticle size ranging from 481.5±26.0 nm to 51.96.4 nm over one order of magnitude, measured by both SEM and single particles tracking techniques, FIG. 5A. The ultra-sonication strategy was utilized for starting milled material and analyzed with SEM (i.e., after wet milling) at sonication durations of 1, 2, 5, 10, and 20 min, FIG. 5B. These sample showed partial sedimentation and therefore were not suitable for analysis with single particle tracking technique. The next samples, which were treated for 30, 40, 50, 60, 70, 80, and 100 min of sonication each, were characterized with a single particle tracking technique which showed continuous reduction of their sizes for prolonged ultra-sonication and a non-sedimenting tendency, FIG. 5C. In addition, the absorption spectrum of each sample showed a gradual decrease in the visible range with the corresponding increase in UV, FIG. 5E. It is worth mentioning that all samples contained the same amount of strontium aluminate material, normalized by an even absorption density at 400 nm. FIG. 5D shows a clear difference in the absorption spectra for smaller particles. Furthermore, measured emission spectra of the samples revealed an 11 nm-blue shift in PL from the wet milled sample to the post milled sample over the time up to 100 min, indicating that the blue shift is also size-dependent, FIG. 5F.

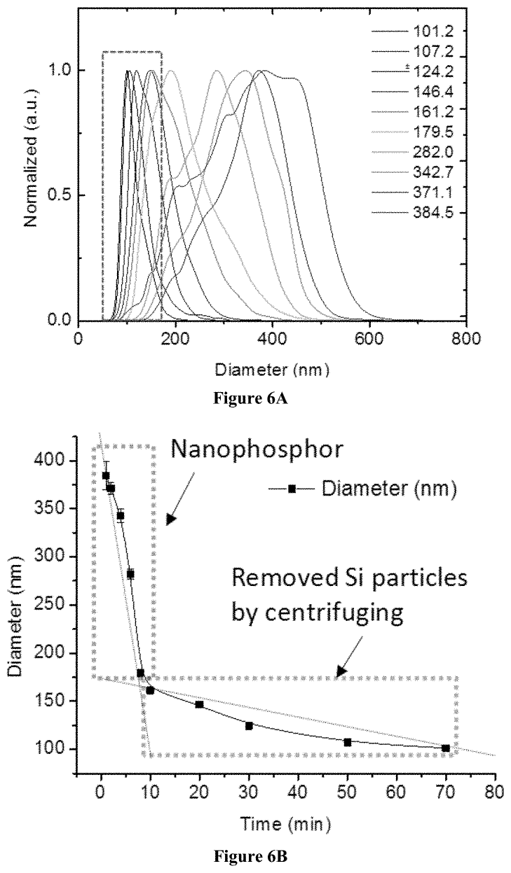

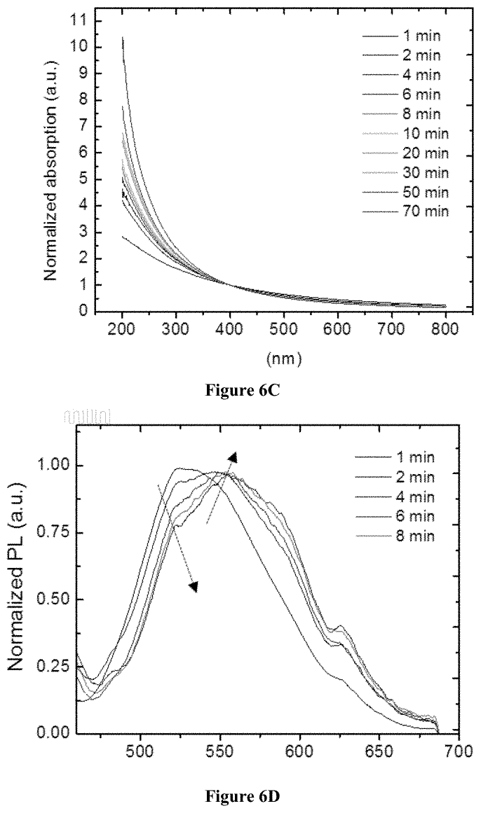

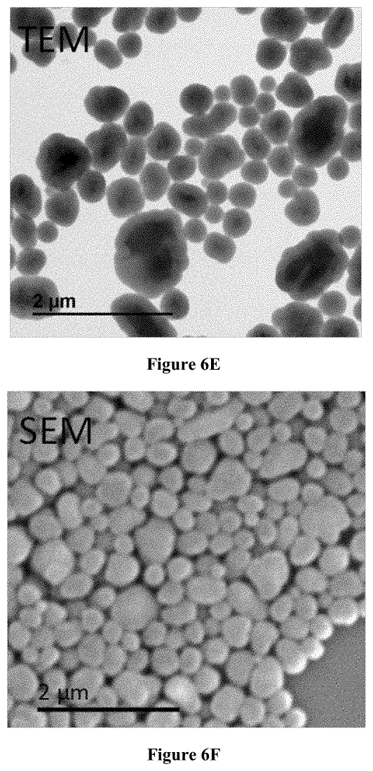

Next, water-suspended Si/SiO₂ coated wet milled strontium aluminate particles materials were centrifuged for time increments ranging from long time of 70 min to short spinning at 1 min. This sorting strategy was designed such that the supernatant was removed after each centrifuging step, and the precipitated material was re-solubilized again in fresh water for the subsequent centrifugation step. After each step of centrifuging, spinning time was reduced and new generation of particles was collected with supernatant. Supernatant removed after each centrifuging step was concentrated and applied for particle analysis with SEM and single particle tracking techniques. This way, the smallest particles, which undergo the slowest sedimentation, are initially collected (at longer centrifuging times) while the large particles undergoing faster sedimentation are collected in the end. Therefore, in the next centrifuging step, an essential portion of the smaller particles is removed already by previous step and a new band of bigger particles can be collected. This strategy allows for the collecting of a particular generation of particles, while avoiding the presence of smaller and larger particles samples together. For this, 500 mg of Si/SiO₂ coated particles dispersed in water was centrifuged for 70, 50, 30, 20, 10, 8, 6, 4, 2, and 1 min respectively. Using the method of precipitate collection described above, our final sample (annotated as centrifuged for 1 min) was also exposed to the nine previous precipitation steps, in which the smaller well solubilized in water particles were removed. Next, dispersions were concentrated with the help of additional centrifugation and characterized with a single particle tracking technique, as all samples showed good stability in water, FIG. 6A. Measurements showed that particles collected by this method have a strikingly different and distinct size distribution, with measured mode values ranging from 110.1±5.0 to 384.5±48.7 nm, FIG. 6A.

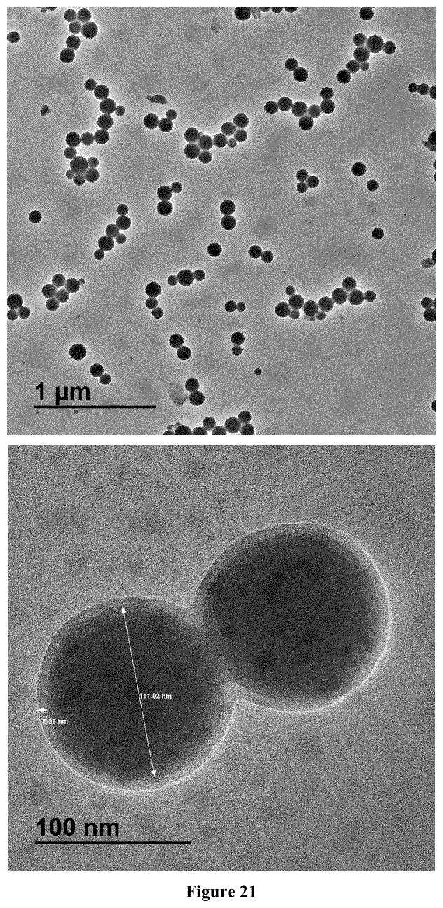

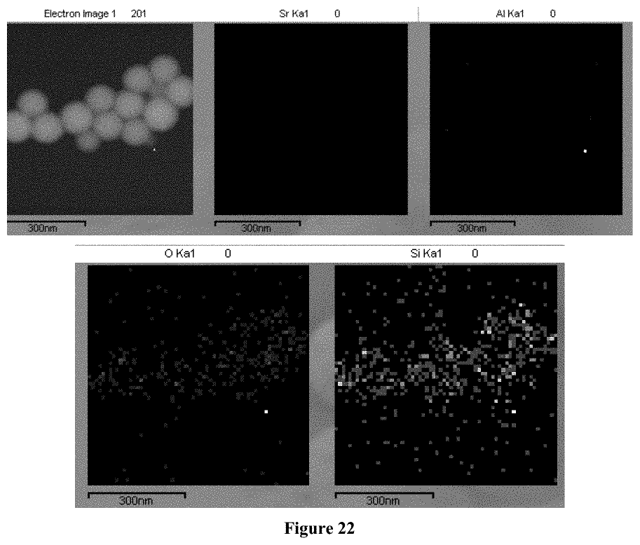

The constructed plot demonstrating the relationship between nanoparticle sizes and centrifugation time clearly indicated two characteristic slopes, FIG. 6B. Particles collected from region I (long time) possessed a very narrow size distribution and close size values. In contrast, the particles collected in region II (short time) had larger sizes, which increased rapidly when the centrifugation time step was shorter (˜10 min). Materials recorded in the first region were assigned to pristine Si/SiO₂ nanoparticles with a narrow size distribution, which was the product of a side reaction formed around the strontium aluminate during Si/SiO₂ coating strategy, FIG. 21. In addition, analysis with TEM equipped with energy-dispersive X-ray spectroscopy clearly confirmed that only the Si and O chemical elements were present, and no phosphors (FIG. 22). It worth mentioning that both TEM and single particle tracking methods sized Si/SiO₂ nanoparticles at ˜110 nm, which validated the precision of these characterization techniques.

Those experiments clearly demonstrate that small silica particles can be gradually and effectively removed by a long centrifuging time, while the Si-coated phosphor nanoparticles remain in the precipitate, due to their larger size and density, which allows them to undergo faster sedimentation during centrifuging. For centrifuging times shorter than 10 min, there is a rapid increase in nanoparticle size, indicating that a different generation of nanoparticles was collected with the supernatant, FIG. 6B. Selected supernatant collected after 2 min of centrifuging was examined with TEM, where no of Si/SiO₂ nanoparticles were observed in the sample, but only strontium aluminate particles with a 100 nm Si/SiO₂ coated shell, FIG. 6E. In addition, SEM measurements show a narrow particle size distribution. Next, the absorption spectra with different samples reveals changes similar to the post-milled phosphor particles with the ultrasonic power discussed above, where smaller particles showed less light scattering at visible range and more in UV region.

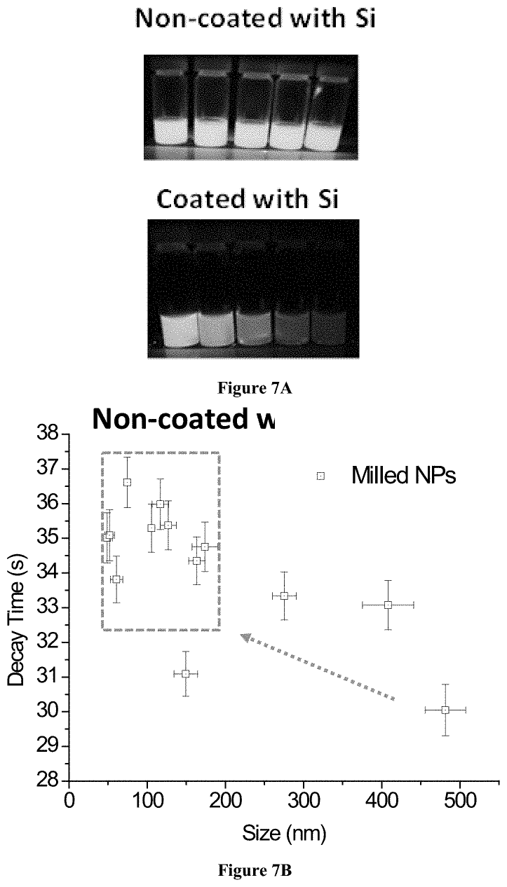

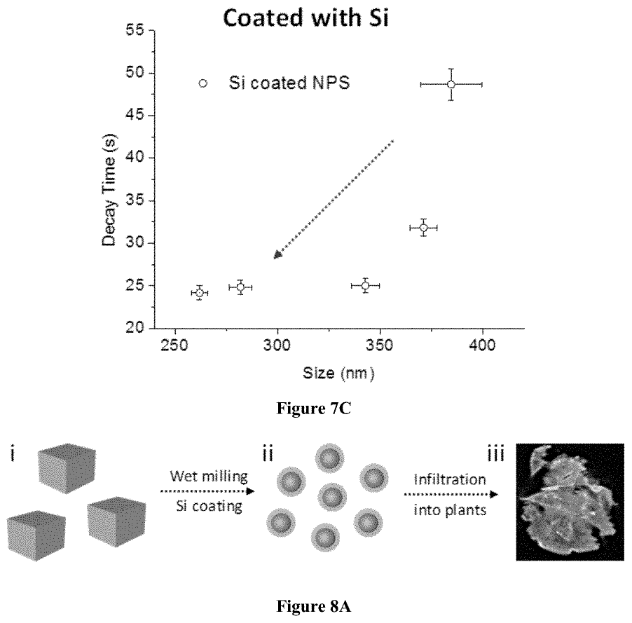

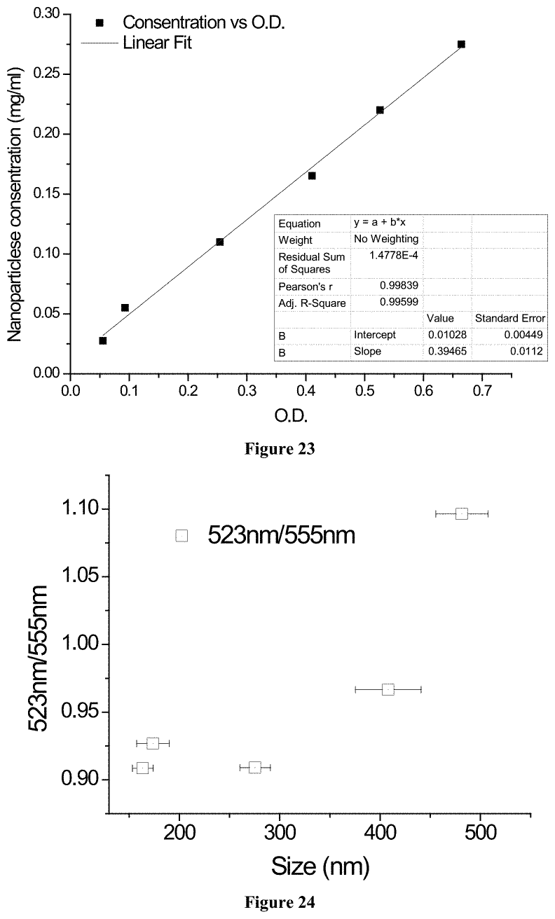

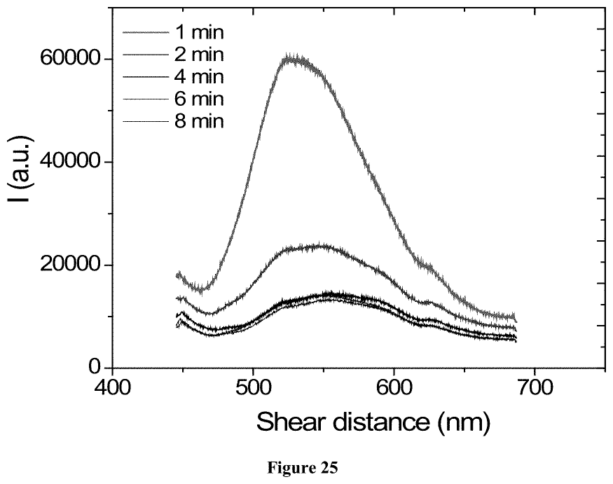

Next, the PL spectra of the samples collected by centrifuging for 1, 2, 4, 6 and 8 min were measured at the same optical density (at 400 nm) correlating with a 1 mg/ml concentration (based on the already measured extinction coefficient, FIG. 23). The PL spectra depicted on FIG. 6D showed a striking dependence of emitted wavelength on a particle having different sizes with a characteristic red shift in emission spectra. Herein, a new peak at 555 nm was measured with a higher intensity than the emission of starting material at 530 nm, FIG. 6D. The ratio of the peak intensities were plotted, measured at 555/523 nm, as a function of particle size in FIG. 24, indicating essential changes for particle size ranging from 262.0±24.2 nm to 384.5±48.7 nm. It is worth mentioning that all particles have a core-shell structure, where the 100 nm Si/SiO₂ shell gives an additional 200 nm increase in measured diameter, and therefore, true strontium aluminate has a ˜60 nm diameter for sample with the smallest particles. As the particle sizes decreases, PL intensity was reduced by 77.3%, FIG. 7C. The pristine mass of strontium aluminate was calculated between those samples based on the core-shell structure, and show that the smallest particles have a smaller mass (by 87.5%) due to the enlarged volumetric mass of Si/SiO₂ shell coating contributing to the total mass of the particle. By taking this in consideration, and re-normalizing the emission intensity per mass on strontium aluminate, the experiments described herein indicate that smaller particles do possess higher PL, FIG. 25. Moreover, post-milled pristine samples generated by ultrasonication, containing different particle sizes, were studied at similar conditions and corresponding decay times were calculated, FIG. 7C. A supporting effect was observed; that the time decay constants of the afterglow samples increase as the particles sizes of non-modified material increased by 22%. Research described herein demonstrates that despite many uncertainties, the emission changes of strontium aluminate particles, which were linked to different synthetic routes; additionally, is highly depended on particle size and particle modification such as Si/SiO₂ coating. Ultrasonic and centrifuging strategies have been developed as facile strategies for sizing of phosphor nanoparticles with characteristic afterglow properties.

Herein, it has been found that it is possible that the emission spectrum of colloidal stable phosphor nanoparticles can shift in both the blue and red directions based on their surrounding environment and specific particle size distribution. Size sorting techniques, and together with absorption spectrum measurements, have clearly shown a relationship between particle size and optical properties, can result in the corresponding changes of PL spectra. Additionally, the optical properties of phosphor nanoparticles can be tailored in advance to set specifications and have promising potential for applications in materials science, biology and sensing equipment.

Light-emitting plants have generated much interest in the society for novel applications such as the illumination of private homes, roads and public areas, and could save up to 8% of total energy consumption. Light-emitting organisms with natural bioluminescence properties can be observed in many marine species, where such function is used in defense mechanisms, burglar-alarming and misleading of predators by rapid light-pulsing. See, for example, Haddock, S. H. D.; Moline, M. A.; Case, J. F. Annu Rev Mar Sci 2010, 2, 443-493 and Rees, J. F.; De Wergifosse, B.; Noiset, O.; Dubuisson, M.; Janssens, B.; Thompson, E. M. J Exp Biol 1998, 201, (8), 1211-1221, each of which is incorporated by reference in its entirety. Similarly, some fungi can emit light used to attract animal grazing, helping to accommodate efficient spore spreading over larger areas. See, for example, Chew, A. L. C.; Desjardin, D. E.; Tan, Y. S.; Musa, M. Y.; Sabaratnam, V. Fungal Divers 2015, 70, (1), 149-187, which is incorporated by reference in its entirety. In addition, fireflies can produce bioluminescence by utilizing their own energy, which can be seen by the naked eye and captured on camera. Living species such as fireflies are capable of conducting a chemical reaction inside their bodies to produce light by the utilization of adenosine triphosphate (ATP) molecules, luciferase and other components such as oxygen and metal ions. In contrast to fungi and other organisms, plants do not have these functions, but instead possess efficient energy storage capabilities, where harvested sun energy can be transferred into biological plant growth via photosynthesis. See, for example, Blankenship, R. E. Blackwell Science Ltd 2008, Print ISBN:9780632043217, which is incorporated by reference in its entirety. The first utilization of plant ATP for light emission was reported in genetically modified Nicotiana tabacum, using the luciferase gene from the Photinus pyralis firefly, which resulted in the report of a glowing plant detected under 24 hours of camera exposure. See, for example, Ow, D. W.; Wood, K. V.; Deluca, M.; Dewet, J. R.; Helinski, D. R.; Howell, S. H. Science 1986, 234, (4778), 856-859, which is incorporated by reference in its entirety. Importantly, luciferin molecule, serving the role of a substrate to luciferase, was introduced into the plant by adsorption through its leaves. Alternative to this strategy, completely autonomously luminescent plants were constructed by the utilization of six lux operon genes inside the chloroplasts of tobacco, leading to observed light emission with a camera exposure time of five minutes. See, for example, Krichevsky, A.; Meyers, B.; Vainstein, A.; Maliga, P.; Citovsky, V. Plos One 2010, 5, (11), which is incorporated by reference in its entirety. However, these genetically modified plants still lack a brightness level sufficient for illumination of surrounded areas or reading. A nanobionic strategy can include utilizing a set of engineered nanoparticles containing luciferase, luciferin and coenzyme A infiltrated into plant leaves to produce a light intensity of over five orders of magnitude brighter than genetically modified plants in modified watercress (Nasturtium officinale) leaves. See, for example, Kwak, S. Y.; Giraldo, J. P.; Wong, M. H.; Koman, V. B.; Lew, T. T. S.; Ell, J.; Weidman, M. C.; Sinclair, R. M.; Landry, M. P.; Tisdale, W. A.; Strano, M. S. Nano Lett 2017, 17, (12), 7951-7961, which is incorporated by reference in its entirety.

In another aspect, the application of nanoparticles in plant science has potential to enhance their functionality or developing functions on-demand, realizing plant life control and optimizing growth conditions. See, for example, Nair, R.; Varghese, S. H.; Nair, B. G.; Maekawa, T.; Yoshida, Y.; Kumar, D. S. Plant Sci 2010, 179, (3), 154-163, which is incorporated by reference in its entirety. For example, it has been shown that carbon nanotubes can help endow chemical detection capabilities in plants, boost their photosynthetic and chemical sensing capabilities, and control the passive transport of these nanoparticles into plant protoplasts by rationally tuning their size and charge. See, for example, Wong, M. H.; Giraldo, J. P.; Kwak, S. Y.; Koman, V. B.; Sinclair, R.; Lew, T. T. S.; Bisker, G.; Liu, P. W.; Strano, M. S. Nature Materials 2017, 16, (2), 264-272, Giraldo, J. P.; Landry, M. P.; Faltermeier, S. M.; McNicholas, T. P.; Iverson, N. M.; Boghossian, A. A.; Reuel, N. F.; Hilmer, A. J.; Sen, F.; Brew, J. A.; Strano, M. S. Nature Materials 2014, 13, (4), 400-408 and Wong, M. H.; Misra, R. P.; Giraldo, J. P.; Kwak, S. Y.; Son, Y.; Landry, M. P.; Swan, J. W.; Blankschtein, D.; Strano, M. S. Nano Lett 2016, 16, (2), 1161-1172, each of which is incorporated by reference in its entirety. Carbon nanotubes can also be used as efficient carrier of genetic cargo into the chloroplasts of mature plants, which cannot be easily realized with conventional methods. See, for example, Lew, T. T. S.; Wong, M. H.; Kwak, S. Y.; Sinclair, R.; Koman, V. B.; Strano, M. S. Small 2018, 14, (44) and Seon-Yeong Kwak, T. T. S. L., Connor J. Sweeney, Volodymyr B. Koman, Min Hao Wong, Karen Bohmert-Tatarev, Kristi D. Snell, Jun Sung Seo, Nam-Hai Chua & Michael S. Strano, 2019, each of which is incorporated by reference in its entirety. Therefore, the exploration of new non-toxic materials, which when introduced can give plants additional functionality, represents a promising direction for the future of plant-nanomaterial research.

In this work, a new strategy based on the development of bright nanobionic light-emitting plants (LEPs) is proposed by utilizing the concept of “light-capacitor”, where infiltrated phosphor particles inside plant leaves are used for light capture and afterglow in the dark. The toxicity of nanoparticles in vivo and the viability of infiltrated plants were studied on lab grown watercress and commercially available plants such as Gerbera Daisy (Bellis perennis) and Basil (Ocimum basilicum). This herein described technology shows, to date, the brightest reported glowing plants from any method with a long afterglow time, which is stable during the plant's lifespan and introduces a new strategy for the application of modified plants in the commercial space.

### Experimental

Particle Preparation.

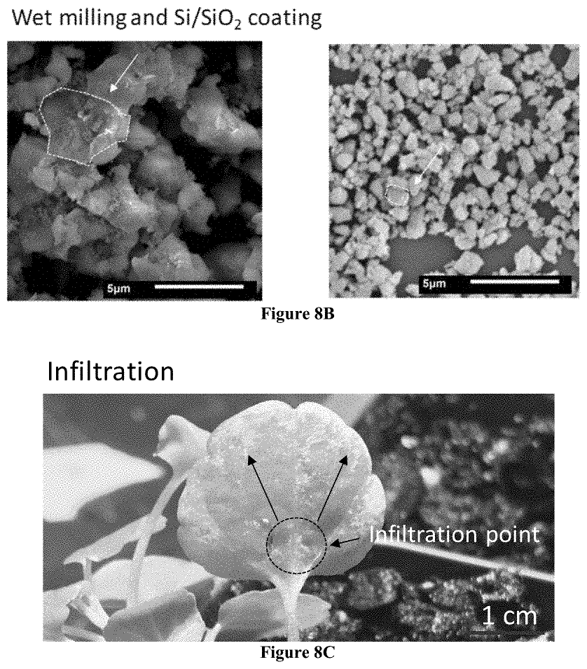

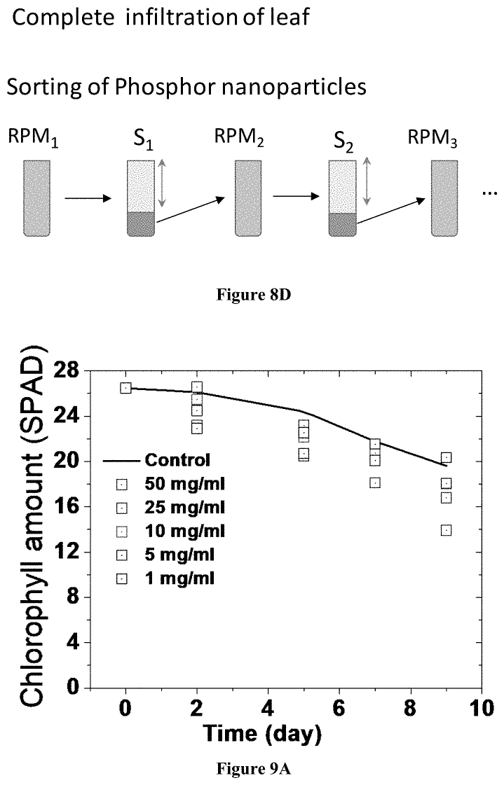

Solid strontium aluminate (SA) phosphor powder containing large particles of up to 3 μm was wet milled for two weeks in ethyl acetate, which resulted in a particle size reduction below one micron, FIGS. 8A and 8B. Furthermore, dissolved milled powder in water showed a pH increase from 7 to 14, due to secondary products related to ion leakage from the metal-oxide matrix. Next, dried milled SA (mSA) material was resuspended in ethanol and modified with a Si/SiO₂ protecting shell (˜100 nm) as described elsewhere. See, for example, Paterson, A. S.; Raja, B.; Garvey, G.; Kolhatkar, A.; Hagstrom, A. E. V.; Kourentzi, K.; Lee, T. R.; Willson, R. C. Anal Chem 2014, 86, (19), 9481-9488, which is incorporated by reference in its entirety. Higher particle solubility in deionized (DI) water and stability against sedimentation was achieved in the Si/SiO₂ protecting shell samples in contrast to the hardly suspended milled material, and as such was used for infiltration in plant leaves.

Nanoparticle Infiltration Toxicity Strategy.

Initially, non-sorted powder of mSA particles was prepared in several dilutions in DI water, ranging from 0.02 mg/ml to 0.3 mg/ml, where the corresponding UV-vis absorbance spectrum was measured and used for calculation of the extinction coefficient at 400 nm, resulting in 0.39±0.01 Optical Density (O.D.)/(mg/ml). This wavelength was chosen since further particle excitations will be realized with a 400 nm light-emitting diode (LED). Furthermore, the impact of infiltrated particles onto plant leaves' longevity was studied by a SPAD-502 chlorophyll meter (Minolta Camera Co., Japan) by measuring the amount of chlorophyll concentrated in Soil-Plant Analyses Development (SPAD) units in the infiltrated leaves over a 10-day period. Several parameters such as particle size, the presence of a Si/SiO₂ protecting shell, pH and concentration were studied and designed in the following way. A few types of phosphor particles samples such as (1) pristine non-milled SA at pH 7, (2) mSA at pH 14, (3) mSA dialyzed over 7 days at pH 7, (4) starting material coated with the Si/SiO₂ at pH 7, and (5) mSA coated with Si/SiO₂ at pH 7 were used for the infiltration of 4 week old watercress plants. Initially, particles were dissolved in buffer (HEPES at pH 7.0, 50 mM) with a 1:1 ratio and infiltrated inside watercress leaves with the help of a tipless syringe. During this step, a gentle pressure was applied to the syringe for a gradual spreading of the particle solution inside the leaves. Importantly, plants were infiltrated by lateral liquid movement along the leaves from a central infiltration point over several centimeters throughout the leaf, FIG. 8D. This infiltration strategy helped ensure that phosphor particles were able to diffuse inside infiltrated leaves far away from the contact point. Secondly, modified leaves were intensively washed with tap water to ensure the removal of any remaining non-infiltrated particles from leaf surfaces at the infiltration points. Moreover, each of the five samples was prepared at several different particle concentrations (1 mg/ml, 5 mg/ml, 10 mg/ml, 25 mg/ml to 50 mg/ml), and infiltrated and measured in five different watercress plants. In a typical infiltration, between 200 μl to 500 μl of sample was used per leaf, and each measurement was averaged over five plants, FIG. 8C. This resulted in the measurement of a total of 150 modified plants, and an additional 30 non-modified plants, which were used as a same-age reference of the modified plants. These control plants were present in each batch of sample and kept at the same growing conditions. All measurements of each of the 180 plants were performed in the same day.

Leaves Longevity Analysis by Chlorophyll Concentration Measurements.

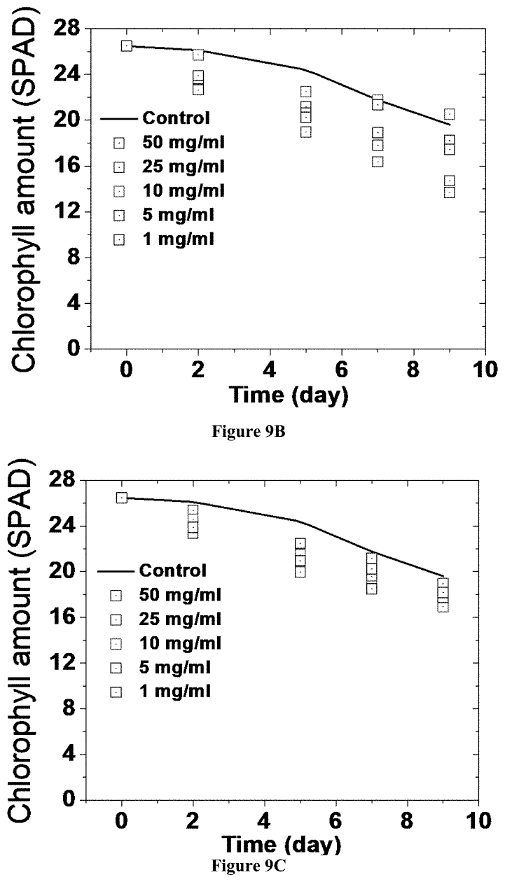

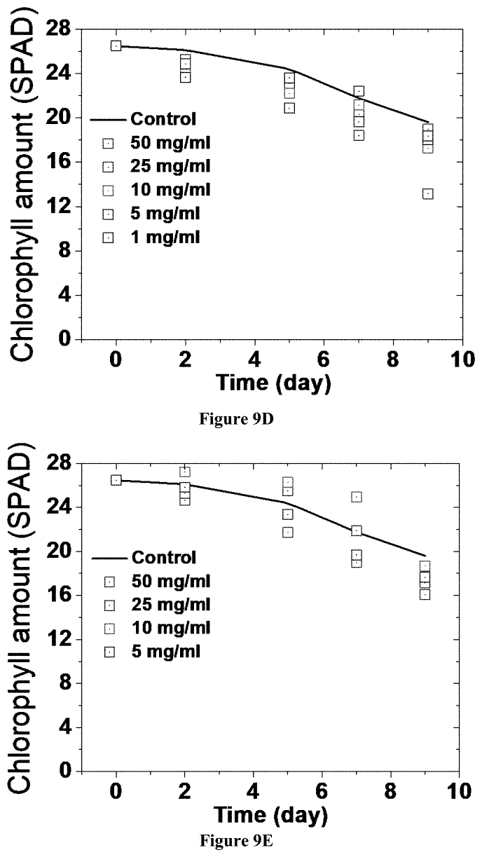

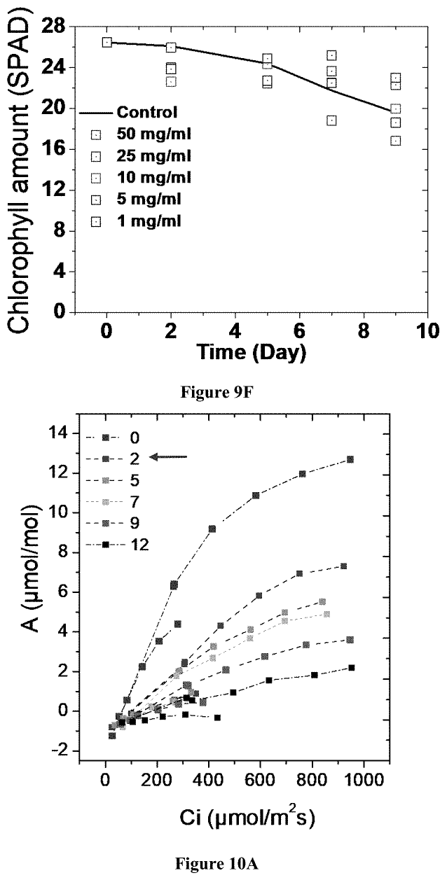

Recorded chlorophyll concentration measurements lent us insight into the effect of particle size, particles concentration and pH on plant health. It is worth mentioning that the measured chlorophyll amounts (SPADs) in the reference unmodified same-age plants showed about 20% reduction over 10 days, due to plant aging. Firstly, the infiltrated SA pristine (FIG. 9A) and Si/SiO₂ coated SA particles (FIG. 9D) showed a harmful effect to the plants as reflected by the decrease in chlorophyll level. The large particles were the most difficult infiltrate, leading to many trials and, therefore, multiple infiltration points which resulted in damage to plant leaves. These infiltration difficulties can be explained by the large particle size of the infiltrated sample since the observed opened stomata pore size is only about 1 μm, which can impede the movement of large particles past the stomata pores of their blocking.

Next, infiltrated mSA particles at pH 14 showed a drastic reduction of chlorophyll amount at all applied concentrations, demonstrating its negative effect as a raw non-modified milled material with high basicity, FIG. 9B. The milled sample neutralized to pH 7 by adding acetic acid also resulted in a reduction of chlorophyll concentration, FIG. 9C. Moreover, the same sample which was neutralized to pH 7 by 3-week dialysis showed a similar damaging role to the plants' leaves, FIG. 9E. In contrast, Si/SiO₂ coated mSA particles demonstrated efficient particle infiltration inside plants leaves and reduced damaging effect, FIG. 9F. For example, infiltrated particle concentrations of 1 mg/ml, 5 mg/ml and 10 mg/ml showed no effect on the chlorophyll concentration over time with respect to non-modified plants.

Li-Cor Measurements for Photosynthetic Activity Study.

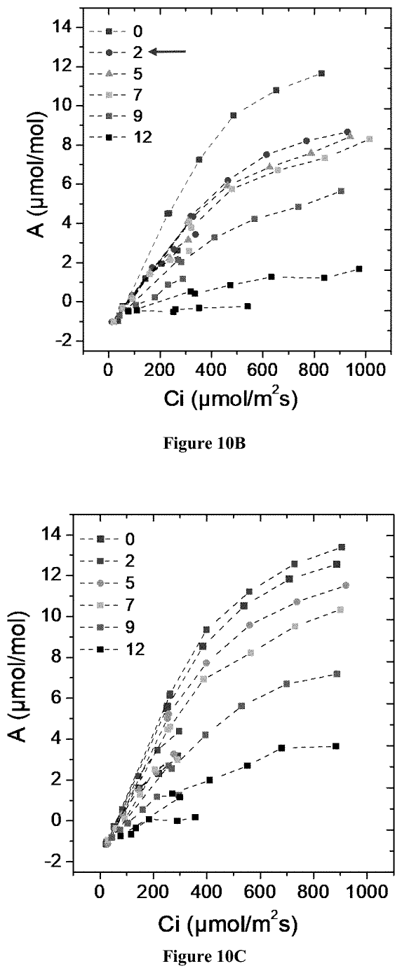

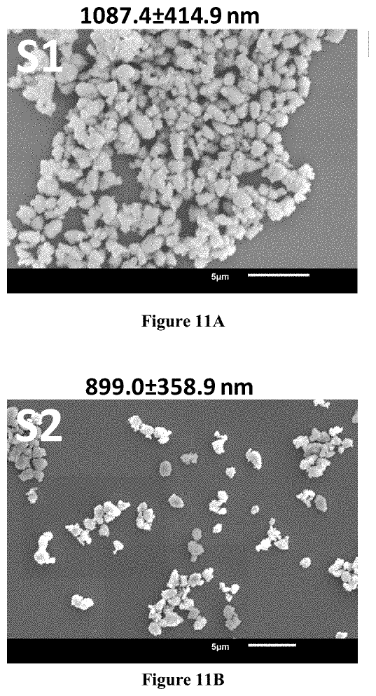

Inspired by minor denaturation properties of Si/SiO₂ modified mSA samples, photosynthesis of modified and unmodified plants was studied in more detail with the Li-Cor characterization technique. Here, watercress plants leaves were infiltrated with the 10 mg/ml mSA phosphor particles in the three-week old watercress plants. One plant infiltrated with buffer and one intact watercress plant were studied as biological references. All seven plants were measured immediately after infiltration and subsequently every two or three days. The net CO₂ assimilation rate (A), as function of internal CO₂ concentrations (Ci) based on reference CO₂ concentrations of 1200, 1000, 800, 600, 400, 300, 200, 100, 50 and 0 μmol·mol⁻¹, a light set point of 900 μmol·m⁻²·s⁻¹, and relative humidity of 50%, FIG. 10A. The determined values of A for watercress are typical for plants with a C3 carbon fixation mechanism which results in high assimilation values. See, for example, Evans, J. R. Oecologia 1989, 78, (1), 9-19, which is incorporated by reference in its entirety. The measured A values show plants' ability to incorporate adsorbed carbon (from CO₂) into larger metabolic carbon pathways such as the Calvin cycle. Watercress leaves measured before infiltration and one week after showed a 20% reduced activity, consistent with the reduction in chlorophyll concentration measurements. At the same time, plant leaves infiltrated with buffer resulted in a similar 15% reduced functionality in comparison to non-modified plant, indicating that mechanical damage of plants leaves during particles infiltration is the key limiting factor, FIG. 10B. All measurements were compared to control experiment of non-infiltrated plant, FIG. 10C.

Sorting of mSA.

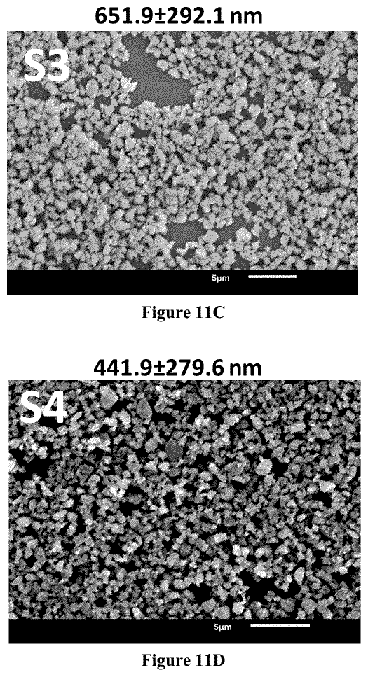

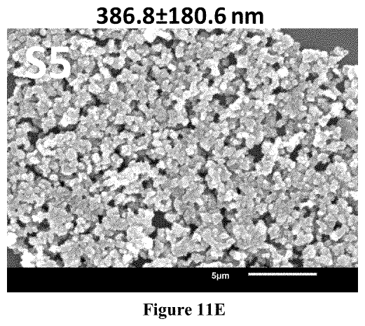

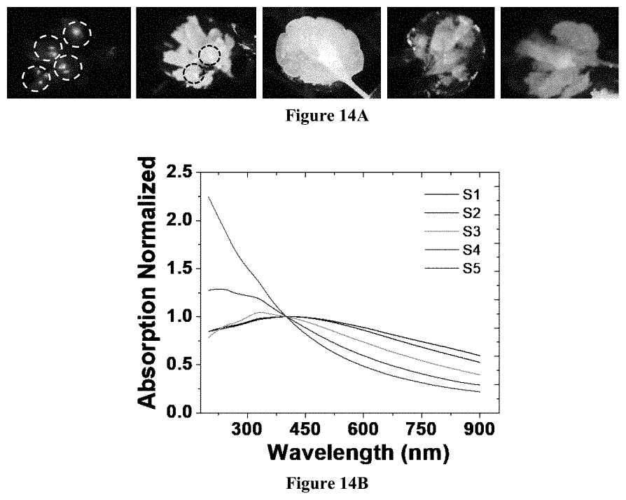

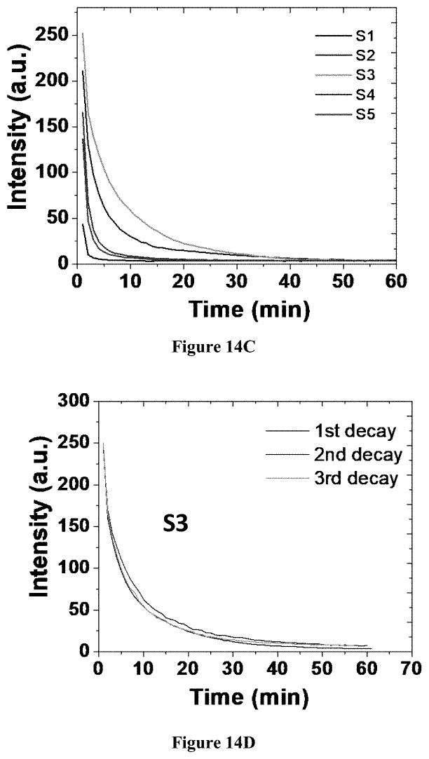

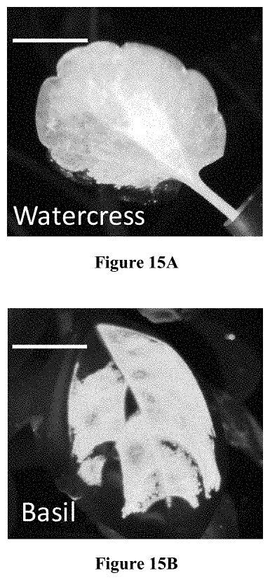

Inspired by the fact that large particles showed a harmful effect on plant leaves, several steps of size sorting via centrifugation were applied to Si/SiO₂ coated mSA particles, FIG. 8D. Initially, Si/SiO₂ coated mSA particles were centrifuged at 4000 rpm for 1 minute, which resulted in the precipitation of larger particles, while the remaining supernatant was removed, concentrated and labeled as the first (Si) sample. Next, the pellet was re-suspended in fresh DI water and centrifuged again, for the same amount of time at a lower speed (3000 rpm) where the resulting supernatant was removed again and treated as the second (S2) sample. This procedure was repeated for 2000, 1000 and 500 rpm rotation speeds leading to S3, S4 and S5 samples respectively. All collected samples were characterized with scanning electron microscopy (SEM) for size distribution resulting in 386.8±180.6 nm, 441.9±279.6 nm, 651.9±292.1 nm, 899.0±358.9 nm and 1087.4414.9 nm respectively, confirming different size distributions due to post-processing. The samples were labeled as S1-S5 respectively, FIGS. 11A-11E. The spectra of the sorted samples were measured and compared, FIG. 14B. Drastic differences in these spectra can be attributed to the particle size-effect associated with Mie light scattering. See, for example, Palkar, S. A.; Ryde, N. P.; Schure, M. R.; Gupta, N.; Cole, J. B. Langmuir 1998, 14, (13), 3484-3492, which is incorporated by reference in its entirety. It is worth mentioning that typical watercress plant leaves require several infiltrations to functionalize the whole area of the leaf. In addition infiltrated particles could not diffuse across leaf sections separated by veins, which prevent their free movement over the entire leaf area. However, sample S3 showed a very rapid infiltration characteristic and prolonged diffusion in all directions of the leaf, with particles penetrating as deep as a plant's stem over 5 cm distance from a single infiltration contact, FIG. 14A. Observed rapid infiltration phenomena can be linked to particle size and charges properties resulting in their propagation inside spongy mesophyll regions and interaction with cell walls of watercress studies later herein.

Confocal Imaging of Strontium Aluminate Particle Distribution Inside Plant Leaves.

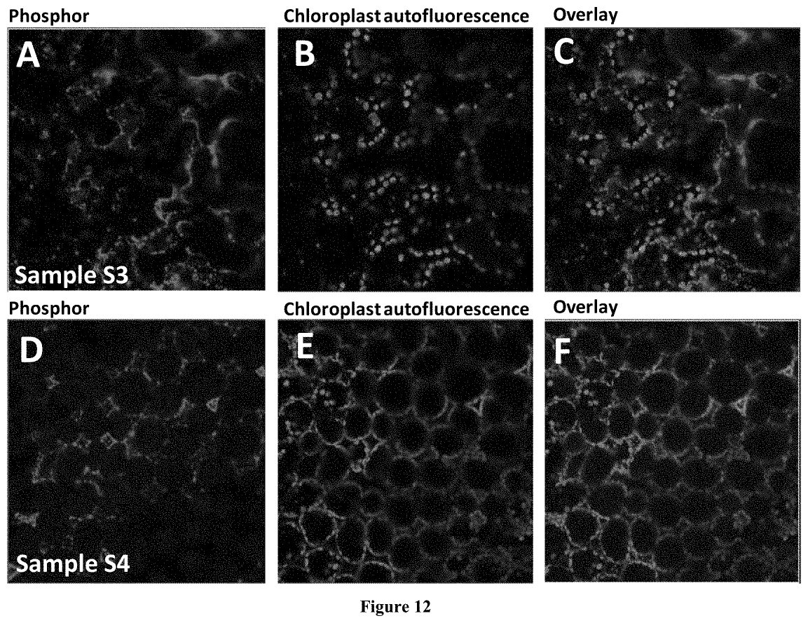

Next, inspired by plant stability against Si/SiO₂ modified mSA particles, infiltrated watercress leaves with the 50 mg/ml solution were imaged by a fluorescence confocal microscope, FIG. 12. In more detail, one leaf was infiltrated with strontium aluminate nanoparticles while the second leaf remained pristine. Images at 620-700 nm (autofluorescence of chlorophylls, FIG. 12 panel B, panel E) were obtained with 632 nm excitation and 510-550 nm emission (phosphorescence, FIG. 12, panel C, panel F) of mSA particles with 488 nm excitation from sample S3 and S4. Optical images indicated that the described above infiltration strategy resulted in homogeneous particle distribution inside the watercress leaf, FIGS. 11A and 11D. This observation supports LEEP theory since the particles' charge (0.57±0.21 mV) and sizes are below the threshold required for passive penetration into the plant cells¹¹. See, for example, Wong, M. H.; Misra, R. P.; Giraldo, J. P.; Kwak, S. Y.; Son, Y.; Landry, M. P.; Swan, J. W.; Blankschtein, D.; Strano, M. S. Nano Lett 2016, 16, (2), 1161-1172, which is incorporated by reference in its entirety.

Scanning Electron Microscopy.

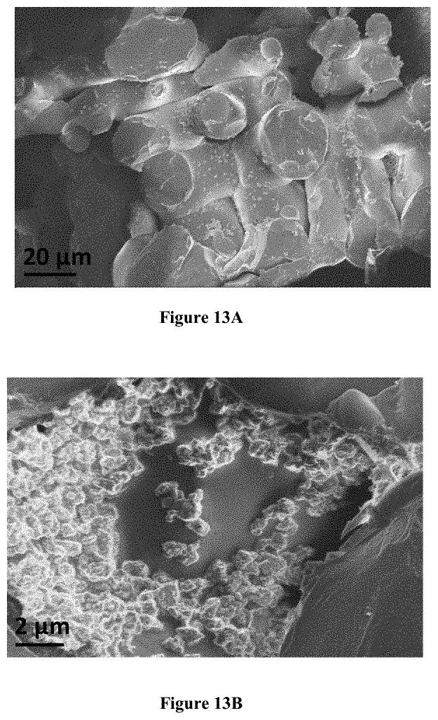

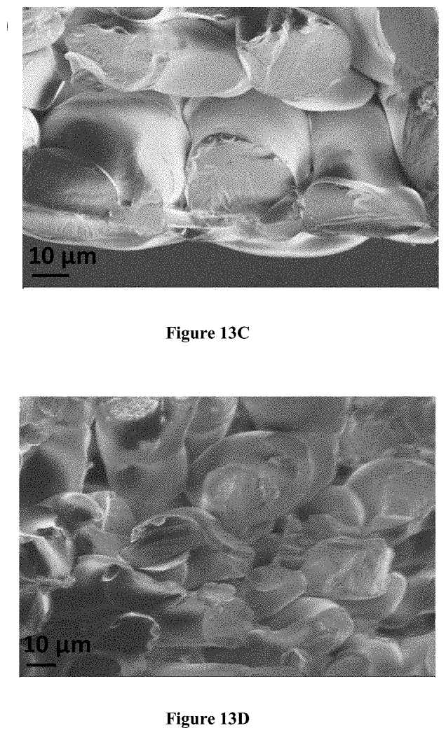

Further studies on leaf tissue integrity was conducted with Cryo scanning electron microscopy (CryoSEM). Initially, one watercress leaf was infiltrated with a 50 mg/ml solution of particles one day before the measurements while the second leaf was non-modified. After the leaf was cut, both modified and non-modified leaves were instantaneously frozen and studied under the same conditions, from a top and cross section view. Measurements revealed the presence of nanoparticles in the infiltrated watercress leaf in the area of sponge mesophylls, FIGS. 13A-13B. Detailed images show that particles homogeneously occupy open cavities inside the plant leaf in the lower epidermis region (the same side as the infiltration). In contrast, watercress leaf infiltrated with buffer showed no particles with a similar mesophyll structure, FIG. 13C. The non-infiltrated leaf revealed the smooth structure of a mesophyll, characteristic of a leaf of a pristine plant, FIG. 13D.

Optical Decay Measurements.

Each sample was infiltrated into 3-week-old watercress leaves as described previously with a final particle concentration of 50 mg/ml, FIG. 14A. It is worth mentioning that infiltrated leaves were extensively washed with tap water to remove non-specifically attached particles from the leaf surface. Afterwards, each leaf was dried and used. Interestingly, only sample S3 with sizes 651.9±292.1 nm showed penetration into the stem of watercress plants, and the sample with S5 (1087.4±414.9 nm) showed heterogeneous character, where a small portion of particles was propagated along the leaves and larger particles remained at the infiltration points, FIG. 14A. This supports our earlier observation that larger particles cannot be diffuse deep into a leaf due to stomata or plant leaf porosity size selective size cutoff, which can be realized only under extensive pressure, leading to leaf damage and overall lower brightness. Samples S5 and S2 with the smaller particle sizes of 386.8180.6 nm and 441.9±279.6 nm respectively showed efficient infiltration in the entire leaves, albeit with reduced brightness, since they have reduced volume-to-surface area ratio which an lead less activity of the particles. Each modified leaf was exposed shortly to a 10 W light-emitting diode for 1 minute with the maximum power intensity at 400 nm (O.D. of samples was fixed to this absorption wavelength), FIG. 14B. Next, the emission phosphorescent intensity of the particles infiltrated inside plant leaves was recorded on camera for one hour, where at each minute a single picture was taken under 30 s exposure, FIG. 14C. Each leaf in triplicate were measured to demonstrate reproducibility and material stability inside the plants after multiple exposure to 400 nm LED, FIG. 14D.

Modification of Older Commercially Available Plants.

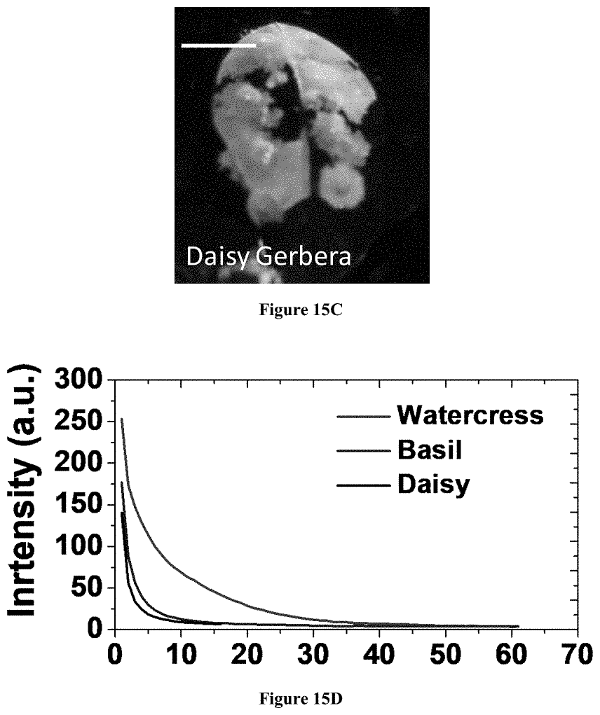

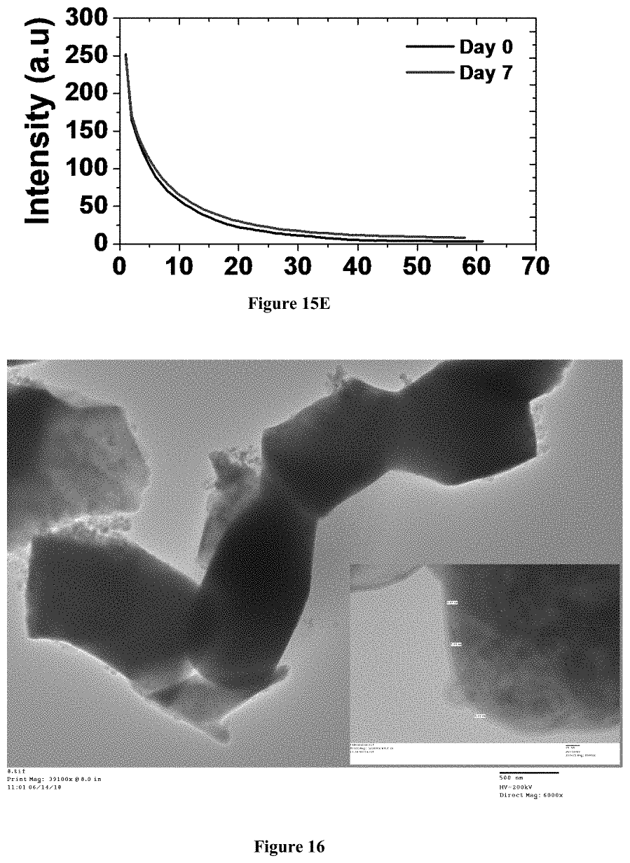

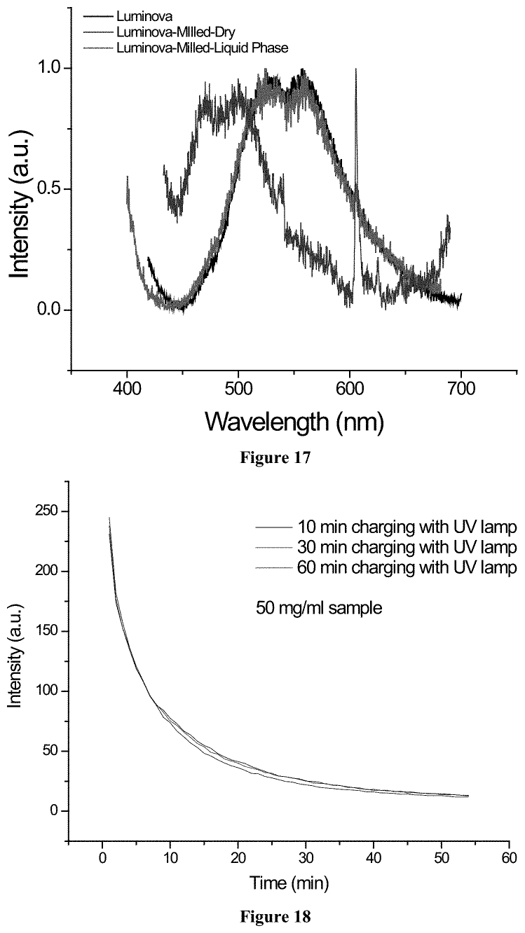

Commercial plants such as Gerbera Daisy and Basil (FIGS. 15A-15C) were infiltrated with 50 mg/ml Si/SiO₂ coated mSA particles and studied for post emission decay. Plants were infiltrated in the same way as discussed before with a goal to produce homogeneous coverage inside plant leaves. The decay intensity of phosphorescence after identical LED charging showed a clear difference between those plant species; watercress possess the brightest intensity while Gerbera Daisies showed less intense phosphorescence after similar excitation conditions, FIG. 15D. The measured chlorophyll amount revealed that watercress resulted in 31.9±1.3, Basil in 22.9±0.3 and Daisy Gerbera in 60.6±0.4 SPAD respectively, emphasizing that the observed phosphorescence effect might be depended on both the chlorophyll concentrations and structure of plants leaves. Due to plant type differences, the internal leaf structure can show selective permeability to a certain type of particles and as result, only a certain size of particles with a size below the characteristic cutoff can be infiltrated. The modified watercress measured one week later showed a similar intensity decay characteristic, indicating stability of the plant leaf and the SA particles inside the leaf, FIG. 15E. A slight increase in intensity was observed after one week, which can be explained by the corresponding attenuation of chlorophyll concentration in plant leaves due to aging as described earlier and leading to a better tissue transparency for emission light.

Successful application of modified mSA particles into plants has been for the development of a new generation of light emitting plants based on the concept of a light capacitor. Functionalized plants with phosphorescent particles showed normal activity and functionality relative to non-modified plants of the same age, resulting in the additional properties of fast light capture and extended afterglow under a rapid excitation with a 400 nm light source. Modified plants have the potential for development of light sensitive sensors in planta. Such a strategy opens new opportunities for the study of sustainable platforms for the new generation of LEPs.

The new set of techniques and an operational theory that is termed as Plant Nanobionics employs a strategy utilizing nanoparticles to engineer living plants with new functionality such as Light Emitting Plant (LEP). The current work introduces an additional nanoparticle designed to augment plant light emission in the form of strontium aluminate nanoparticles as nanophosphor elements. These nanoparticles can absorb and re-emit generated light at longer times, increasing the duration of light emission. Moreover, such nanophosphores can also scavenge additional energy from solar fluence, increasing and augmenting total light emission from the plant. Infiltrated strontium aluminate particles showed homogeneous distribution inside plants leaves in spongy mesophyll region without penetration inside plants cell, preserving their intact structure, as well as efficient particle infiltration deep into the plant's structure. Investigation on the photosynthetic activity of modified plants confirmed their non-toxic biological effect with minor reduction of chlorophyll amount comparable to non-modified plants related to mechanical damaging during particle infiltration.

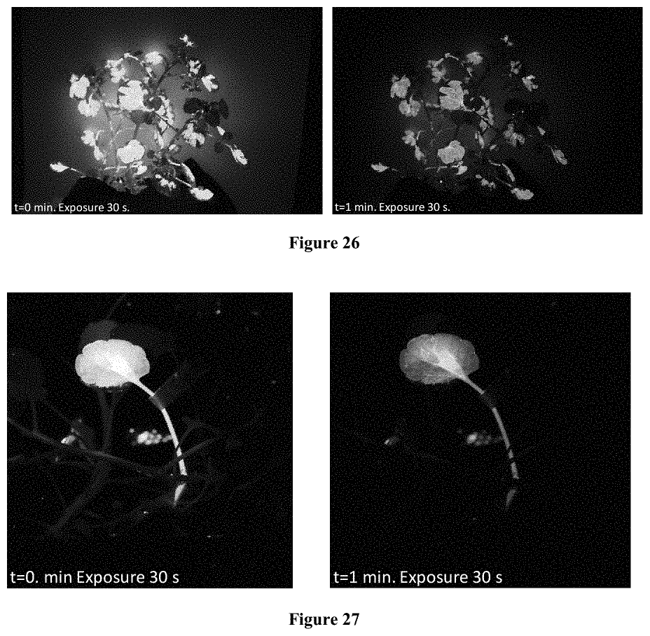

In the current experiment, modified watercress (Nasturtium officinale) plants leaves with phosphorescent particles excited with 400 nm light for 5 seconds demonstrate prolong green light emission up to several hours with monotonic intensity decay properties. Moreover, frequent (each 10-30 min) exposure to blue light revealed no harmful effects on plants functionality as well. Performed experiments in the prototype plants demonstrated that rapidly harvested light by infiltrated particles could emit light for prolonged time, rendering the use of these nanoparticles as biocompatible light capacitor. The current study shows that infiltrated particles in plants leaves are evenly distributed over the entire leaf area (FIG. 26) with bright emission intensity. Additionally, our studies demonstrate that applied particles into plants leaves can enter the plant's stem for several centimeters deep, confirming their efficient mobility inside living systems (FIG. 27). While the proof-of-concept experiment is shown here, the final plants will contain several nanoparticles in order to reach the maximum lighting efficiency by utilizing energy from plant's ATP and sunlight harvested from both visible and infrared spectra.

### Methods

Phosphor Milling.

100 g of Strontium Aluminate powder was dispersed in 100 ml of ethyl acetate in a 300 mL ceramic milling jar (U.S. Stoneware Roalox Alumina-Fortified Grinding Jar) with small and large zirconia cylinders as the grinding media in the rotation mill (Labmill-8000, 1 Tier, 115/220V VAC) for 7-14 days. Afterwards, the remaining ethyl acetate was removed under nitrogen gas, and dried samples were used for further modification steps.

Si/SiO₂ Coating of Milled Phosphor Nanoparticles.

500 mg of milled strontium aluminate powder was suspended in 100 ml of anhydrous ethanol in a 100 mL glass vial and sealed with a plastic cap. Next, 25 ml of MilliQ water and 3.3 ml of tetraethyl orthosilicate (TEOS) were added to the solution and sonicated (Branson 2800 Sonicator) for 10 min, followed by the addition of 12 ml of aqueous ammonium hydroxide (28-30%). After, the sample was stirred for 12 hours at 300 RPM at room temperature (22° C.). The reaction was terminated by the addition of additional water followed with a 1.5 hour centrifugation step (Eppendorf 5810 R at 4000 RPM), after which the particle precipitate was resuspended in fresh DI water. This procedure was repeated several times.

Ultrasonic Post Milling of Phosphor Nanoparticles.

Wet milled strontium aluminate powder (100 mg) was placed in a 20 ml glass vial and ultra-sonicated at different times ranging from 1 to 100 min at Qsonica Sonicators at a power setting of 125 Watt, 20 kHz frequency and amplitude set at 55%. The tip nozzle 435-A was placed deep into solution, close to the bottom of the glass vial, while the bottom part was placed in the water. Milled samples were further analyzed with the UV-vis absorption spectrophotometer Shimadzu, UV-3101PC UV-VIS-NIR scanning spectrometer.

Sorting of Si/SiO₂ Coated Nanophosphor.

Freshly coated milled nanoparticles with Si/SiO₂ shell (500 mg) were gently sonicated for 1 min in Branson 2800 Sonicator and spun down in centrifuge Eppendorf 5810 R at 4000 RPM at different times ranging from 70 min to 1 min respectively. After each sonication step, the supernatant (containing non-measured sedimentation particles) was removed and concentrated with Millipore filter with 100 K molecular cutoff Amicon Ultra-15 Centrifugal Filter Units.

Characterization of Nanoparticles.

Particles that had been milled and size sorted by centrifugation were subject to a surface charge, or zeta potential measurement, which was averaged over 10 runs with the help of phase analysis light scattering zeta potential analyzer (PALS) (NanoBrook ZetaPALS Potential Analyzer, NY, U.S.A.). The nanoparticle sizes were analyzed with the NanoSight LM10 (NanoSight Ltd., Amesbury, United Kingdom) and the scanning electron microscope (SEM) JSM-6010LA InTouchScope (JEOL Ltd.).

Photoluminescence Measurements.

The phosphorescence spectra were measured in a transmission configuration with an incident angle of 45 with respect to the collection pathways. 365 nm light from a light-emitting-diode (Thorlabs, M365L2) was focused on the phosphor dispersion in a glass vial through a condenser lens under continuous stirring. The typical excitation power was ranged between 1 mW and 100 W and the exposure time was 500 ms. The phosphorescence signal was collected through a spectrometer and an N₂-cooled charge-coupled device camera (Princeton Instruments, PyLoN).

Watercress Plants.

Watercress plants were kept at room conditions in dark boxes (made out of wood), where each box was equipped with two types of LED lamps required for supporting plants growth and selective excitation of infiltrated phosphor particles respectively. The first lamp, which is a growth lamp LED with absolute daylight spectrum (Miracle LED Absolute Daylight Spectrum Grow Lite) was switch “on” for 10 hours during each night for realization of plants growth, while the second UV LED lamp with emission intensity at 380-400 nm light was used in blinking mode (LED Black Lights Bulb, 7W A19 E26 Bulb, UVA Level 385-400 nm, Onforu Lights) during a day. Applied UV LED lamp can do selective excitation of phosphor particles inside plants leaves with no damage to the plants. In such experiment, the UV LED lamp was used to illuminate watercress plants each 1 minute for 5 s. Such short excitation allows human to see (through fabricated 5 cm in diameter round opening in box) green light emission coming from infiltrated leaves till the next excitation by naked eyes or even capture pictures of glowing plants on a cellphone camera. Performed experiments over 3 weeks revealed intact structures of the plants with no damage due to UV light or artificial growing conditions, indicating this setup as satisfactory for applications on exhibition for prolonging the time of several weeks.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed is:

1. A light emitting plant, comprising:
a plant structure and a light capacitor in a portion of the plant structure.

2. The light emitting plant of claim 1, wherein the light capacitor is phosphorescent.

3. The light emitting plant of claim 1, wherein the light capacitor is a phosphorescent microparticle or nanoparticle.

4. The light emitting plant of claim 1, wherein the light capacitor scavenges additional energy from solar fluence, increasing and augmenting total light emission from the plant.

5. The light emitting plant of claim 1, wherein the plant further includes a second emissive component.

6. The light emitting plant of claim 1, wherein the light capacitor is a phosphorscent nanoparticle including a strontium aluminate.

7. The light emitting plant of claim 1, wherein the light capacitor is distributed inside plants leaves in spongy mesophyll region without penetration inside plants cell.

8. The light emitting plant of claim 1, wherein the light capacitor is distributed inside the plant's stem.

9. The light emitting plant of claim 1, wherein the light capacitor is a coated particle.

10. The light emitting plant of claim 9, wherein the silica coated particle is a silica coated strontium aluminate.

11. The light emitting plant of claim 1, wherein the silica coated strontium aluminate is phosphorescent.

12. A plant, comprising:
a light generator; and
a light capacitor for upconverting absorbed light to a wavelength absorbed by the light generator,
wherein the light generator and the light capacitor are within a structure of the plant.

13. A method of generating light from a plant comprising:
generating light in the plant with a light capacitor within the plant.

14. The method of claim 13, further comprising:
storing energy from the light in the light capacitor; and
releasing photons from the light capacitor, wherein the light capacitor is in a plant.

15. The method of claim 13, wherein the light capacitor includes a phosphorescent material.

16. The method of claim 15, wherein the phosphorescent material has an emission lifetime of greater than 1 millisecond.

17. The method of claim 13, wherein the light capacitor includes an up-conversion material.

18. The method of claim 17, wherein the up-conversion material includes a metal porphyrin and anthracene.

19. The method of claim 17, further comprising emitting light from a light generator.

20. The method of claim 19, wherein the light generator includes luciferase-luciferin.
